(19) United States
(12) Patent Application Publication
(10) Pub. No.: US 2023/0384286 A1
(43) Pub. Date: Nov. 30, 2023

(54) Systems and Methods for Oxidizing Phenolic Cannabinoids with Fuel Cells

(71) Applicant: ElectraTect, Inc., Phoenix, AZ (US)

(72) Inventors: Evan Rashied Darzi, Phoenix, AZ (US); Di Huang, Phoenix, AZ (US); Christina Rae Forbes, Phoenix, AZ (US)

(73) Assignee: ElectraTect, Inc., Phoenix, AZ (US)

(21) Appl. No.: 18/317,873

(22) Filed: May 15, 2023

Related U.S. Application Data
(60) Provisional application No. 63/364,701, filed on May 13, 2022.
(60) Provisional application No. 63/375,523, filed on Sep. 13, 2022.

(51) Int. Cl. G01N 33/487; G01N 33/497

(52) U.S. Cl. CPC G01N 33/48714; G01N 33/497

(57) ABSTRACT

Systems and methods for oxidizing phenolic cannabinoids with fuel cells are described. The oxidation processes for phenolic cannabinoids and/or Δ⁹-THC can be detected and the concentration of phenolic cannabinoids and/or Δ⁹-THC can be reported directly with fuel cells. Many embodiments provide integrating cannabinoid fuel cells into marijuana breathalyzer devices.

### CROSS-REFERENCE TO RELATED APPLICATIONS

The current application claims the benefit of and priority under 35 U.S.C. § 119 (e) to U.S. Provisional Patent Application No. 63/364,701 entitled “Systems and Methods for Oxidizing Phenolic Cannabinoids with Fuel Cells” filed May 13, 2022, U.S. Provisional Patent Application No. 63/375,523 entitled “Systems and Methods for Oxidizing Phenolic Cannabinoids with Fuel Cells” filed Sep. 13, 2022. The disclosures of U.S. Provisional Patent Application No. 63/364,701, U.S. Provisional Patent Application No. 63/375,523 are hereby incorporated by reference in their entirety for all purposes.

### FIELD OF THE INVENTION

The present invention generally relates to systems and methods for tetrahydrocannabinol oxidation with fuel cells; and more particularly to systems and methods for direct detection of tetrahydrocannabinol with fuel cells.

### BACKGROUND OF THE INVENTION

Marijuana has been used as a recreational drug for many millennia, and has become one of the most commonly used drugs in the United States and many other countries. Marijuana and other cannabinoid products have been considered illicit substances in many countries. However, there have been notable efforts to legalize these drugs for recreational purposes, which have led to the legalized use of marijuana. With the easement of laws and enforcement concerning marijuana, there has been a growing interest in safety, especially when it comes to driving motorized vehicles, akin to long-standing concerns about driving under the influence of alcohol. Marijuana can have negative impacts on spatial and temporal judgments. A reliable and easy-to-use system to detect recent marijuana use is necessary.

A fuel cell is an electrochemical device that converts the chemical energy of a fuel (such as hydrogen) and an oxidizing agent (such as oxygen) into electricity through a pair of redox reactions. Fuel cells may require a continuous source of fuel and oxygen (usually from air) to sustain the chemical reaction. Fuel cells can produce electricity continuously for as long as fuel and oxygen are supplied.

### BRIEF SUMMARY

Systems and methods in accordance with various embodiments of the invention enable phenolic cannabinoids oxidation using fuel cells. In many embodiments, phenolic cannabinoids can be directly oxidized for detection using fuel cells. Phenolic cannabinoids can be oxidized to their corresponding quinones. A number of embodiments provide cannabinoid fuel cells which can be integrated into cannabinoid breathalyzers. Several embodiments provide fuel cells that can utilize phenolic cannabinoids to generate electricity. In many embodiments, phenolic canabinoids from products including (but not limited to) hemp waste can be oxidized to generate electricity using fuel cells. Examples of phenolic cannabinoids include (but are not limited to) tetrahydrocannabinol (THC or Δ⁹-THC), Δ⁸-THC, cannabinol (CBN), and cannabidiol (CBD). Several embodiments provide that fuel cells can detect the oxidation of phenolic cannabinol including (but not limited to) tetrahydrocannabinol. In some embodiments, the number of electrons transferred during the phenolic cannabinol oxidation can be detected. In many embodiments, the direct oxidation processes of THC can generate tetrahydrocannabinol p-quinone or o-quinone (THCQ or Δ⁹-THCQ). In some embodiments, the oxidation processes can be chemical including (but not limited to) electrochemical processes. THC can be detected in gas phase and/or solution phase with fuel cells in accordance with many embodiments. In several embodiments, the oxidation of THC for detection occurs in real-time. The measurable signals including (but not limited to) current, voltage, power, and total charge, have a linear relationship with THC input in accordance with some embodiments. Certain embodiments provide a higher amount of THC in the input as the fuel can generate a higher output signal.

An embodiment includes a method of oxidizing cannabinoid with a fuel cell comprising:

obtaining a sample from a source;

oxidizing the sample electrochemically using a fuel cell;

analyzing at least one signal of the oxidized sample selected from the group consisting of current, power, current density, power density, and charge; and

identifying if the cannabinoid is present based on the at least one signal of the oxidized sample.

In another embodiment, the sample is either in liquid phase or in gas phase.

In a further embodiment, the sample is a biological sample extracted from an individual and the biological sample is biofluid, tear, saliva, mucus, urine, sweat, blood, or plasma.

In an additional embodiment, the sample is in gas phase and the sample is breath.

In another further embodiment, the fuel cell comprises at least one electrolyte comprising at least one electrolyte salt selected from the group consisting of NBu₄PF₆, NEt₄PF₆, LiPF₆, LiPF₄, NBu₄BF₄, NEt₄BF₄, NBu₄ClO₄, and LiClO₄, dissolved in a solvent selected from the group consisting of an aqueous solvent, an organic solvent, and a mixture of an aqueous solvent and an organic solvent.

In an additional further embodiment, the fuel cell comprises at least one solid electrolyte.

In a further yet embodiment, the at least one electrolyte has a concentration from 0.01 M to 1 M, and the solvent has a volume fraction from 96% to 100%.

In yet another embodiment, the fuel cell comprises a cathode comprising a material selected from the group consisting of a transition metal, a metal oxide, a metal, and a metal alloy.

In another embodiment again, the cathode is supported on a material selected from the group consisting of carbon, carbon black, carbon powder, carbon black powder, graphene, graphite, fullerene, nanotube, and carbon nanotube.

In yet another embodiment, the fuel cell comprises a cathode selected from the group consisting of platinum on carbon cloth, platinum on carbon paper, and platinum and ruthenium on carbon cloth.

In a further embodiment again, the fuel cell comprises an anode comprising a material selected from the group consisting of a transition metal, a metal oxide, a metal, and a metal alloy.

In a yet further embodiment, the anode is supported on a material selected from the group consisting of carbon, carbon black, carbon powder, carbon black powder, graphene, graphite, fullerene, nanotube, and carbon nanotube.

In a further yet embodiment, the fuel cell comprises an anode selected from the group consisting of Ni(OH)₂, Ni(OH)₂ modified with multi-wall carbon nanotubes (MWCNTs), CuO, CuO modified with MWCNTs, glassy carbon electrode, Cu on a carbon support, Pd on a carbon support, Pt on a carbon support, Fe on a carbon support, Pd on a carbon support, Rh on a carbon support, Ni on a carbon support, Ru on a carbon support, Pt and Ni on a carbon support, and Ni(OH)₂ on a carbon support.

In another embodiment yet again, the carbon support is selected from the group consisting of: carbon black, carbon black XC-72, Vulcan XC72, Vulcan XC72R, carbon black powder, and Super P® carbon black powder.

In another further embodiment, the fuel cell comprises a platinum on carbon cloth cathode and a Ru on a carbon support anode; or a carbon cloth cathode and a Ni(OH)₂ modified with MWCNTs anode; or a carbon cloth cathode and a CuO modified with MWCNTs anode; or a carbon cloth cathode and a Ru on Vulcan XC72 anode; or a carbon cloth cathode and a Pt on Vulcan XC72 anode.

In a further yet embodiment, the fuel cell comprises an ion exchange membrane or a proton conducting membrane.

In yet another embodiment, the ion exchange membrane is selected from the group consisting of Nafion® 117, Nafion® 112, Nafion® 212, Xion® PEM, Fumasep® F930, Fumasep® FKB-PK-130, Fumasep® F950, Fumasep® FS950, Fumasep® FKE-50, and Fumasep® FAS-30.

In an additional embodiment again, the fuel cell is a H-cell, a flow cell, or a stack cell.

In yet another embodiment, the fuel cell is configured to be integrated in a breathalyzer.

In a further yet embodiment, the identification is in real-time.

In another further embodiment, the cannabinoid is selected from the group consisting of Δ⁹-THC, Δ⁸-THC, CBN, and CBD.

In a further embodiment again, the fuel cell is part of an energy production process.

Another additional embodiment further comprises calibrating the fuel cell to establish a base line signal.

In yet another embodiment, the identification of cannabinoid outputs a cannabinoid concentration in the sample.

In a further embodiment again, the at least one signal of the oxidized sample has a linear relationship with the cannabinoid concentration.

In yet another embodiment again, the cannabinoid is Δ⁹-THC and the oxidized sample is Δ⁹-THCQ.

Another embodiment includes a cannabinoid fuel cell comprising: a cathode; an anode; an ion exchange membrane; and an electrolyte; wherein the ion exchange membrane is disposed between the cathode and the anode, and the electrolyte is in contact with the anode; and wherein the fuel cell is configured to oxidize a sample electrochemically; analyze at least one signal of the oxidized sample selected from the group consisting of current, power, current density, power density, and charge; and output a cannabinoid concentration from the sample.

In an additional embodiment, the sample is either in liquid phase or in gas phase.

In a further embodiment, the sample is a biological sample extracted from an individual and the biological sample is biofluid, tear, saliva, mucus, urine, sweat, blood, or plasma.

In another embodiment again, the sample is in gas phase and the sample is breath.

In yet another embodiment, the electrolyte comprises at least one electrolyte salt selected from the group consisting of NBu₄PF₆, NEt₄PF₆, LiPF₆, LiPF₄, NBu₄BF₄, NEt₄BF₄, NBu₄ClO₄, and LiClO₄, dissolved in a solvent selected from the group consisting of an aqueous solvent, an organic solvent, and a mixture of an aqueous solvent and an organic solvent.

In a further yet embodiment, the electrolyte is a solid electrolyte.

In another further embodiment, the electrolyte has a concentration from 0.01 M to 1 M, and the solvent has a volume fraction from 96% to 100%.

In yet another embodiment, the cathode comprises a material selected from the group consisting of a transition metal, a metal oxide, a metal, and a metal alloy.

In another embodiment again, the cathode is supported on a material selected from the group consisting of carbon, carbon black, carbon powder, carbon black powder, graphene, graphite, fullerene, nanotube, and carbon nanotube.

In another yet embodiment, the cathode is selected from the group consisting of platinum on carbon cloth, platinum on carbon paper, and platinum and ruthenium on carbon cloth.

In yet another further embodiment, the anode comprises a material selected from the group consisting of a transition metal, a metal oxide, a metal, and a metal alloy.

In an additional embodiment again, the anode is supported on a material selected from the group consisting of carbon, carbon black, carbon powder, carbon black powder, graphene, graphite, fullerene, nanotube, and carbon nanotube.

In a further yet embodiment, the fuel cell comprises an anode selected from the group consisting of Ni(OH)₂, Ni(OH)₂ modified with multi-wall carbon nanotubes (MWCNTs), CuO, CuO modified with MWCNTs, glassy carbon electrode, Cu on a carbon support, Pd on a carbon support, Pt on a carbon support, Fe on a carbon support, Pd on a carbon support, Rh on a carbon support, Ni on a carbon support, Ru on a carbon support, Pt and Ni on a carbon support, and Ni(OH)₂ on a carbon support.

In yet another further embodiment, the carbon support is selected from the group consisting of: carbon black, carbon black XC-72, Vulcan XC72, Vulcan XC72R, carbon black powder, and Super P® carbon black powder.

In a further yet embodiment, the cathode is a platinum on carbon cloth and the anode is Ru on a carbon support; or the cathode is carbon cloth and the anode is Ni(OH)₂ modified with MWCNTs; or the cathode is carbon cloth and the anode is CuO modified with MWCNTs; or the cathode is carbon cloth and the anode is Ru on Vulcan XC72; or the cathode is carbon cloth and the anode is Pt on Vulcan XC72.

In an additional further embodiment, the ion exchange membrane is a proton conducting membrane.

In a further embodiment again, the ion exchange membrane is selected from the group consisting of Nafion® 117, Nafion® 112, Nafion® 212, Xion® PEM, Fumasep® F930, Fumasep® FKB-PK-130, Fumasep® F950, Fumasep® FS950, Fumasep® FKE-50, and Fumasep® FAS-30.

In yet another embodiment, the fuel cell is a H-cell, a flow cell, or a stack cell.

In another further embodiment, the fuel cell is configured to be integrated in a breathalyzer.

In an additional embodiment again, the fuel cell outputs the cannabinoid concentration in real-time.

In a yet further embodiment, the cannabinoid is selected from the group consisting of Δ⁹-THC, Δ⁸-THC, CBN, and CBD.

In a further embodiment again, the fuel cell is part of an energy production process.

Yet another embodiment further comprises a computer system to analyze the at least one signal of the oxidized sample.

In a further embodiment again, the at least one signal of the oxidized sample has a linear relationship with the cannabinoid concentration.

Another further embodiment comprises an anode gas diffusion layer, an anode flow plate, an anode current collector, an anode end plate, a cathode gas diffusion layer, a cathode flow plate, a cathode current collector, and a cathode end plate.

In an additional embodiment yet again, the cannabinoid is Δ⁹-THC and the oxidized sample is Δ⁹-THCQ.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 illustrates a phenolic cannabinoids detection process with a fuel cell in accordance with an embodiment of the invention.

FIG. 2 illustrates a tetrahydrocannabinol detection process using a fuel cell breathalyzer in accordance with an embodiment.

FIG. 3 illustrates the molecular structure of different phenolic cannabinoids and quinoidal oxidation products.

FIG. 4 illustrates the oxidation of Δ⁹-tetrahydrocannabinol (Δ⁹-THC) to corresponding p-quinone and/or o-quinone, Δ⁹-THCQ.

FIG. 5 illustrates a THC fuel cell in accordance with an embodiment.

FIG. 6 illustrates a THC fuel cell stack in accordance with an embodiment.

FIG. 7 illustrates a linear response of a THC fuel cell at various THC concentrations in accordance with an embodiment.

FIGS. 8A-8C illustrate THC fuel cell performance with different anode materials in accordance with an embodiment.

FIG. 9 illustrates a THC H-cell in accordance with an embodiment.

FIG. 10 illustrates a collection of power density vs current density of various electrolyte salts for THC fuel cells in accordance with an embodiment.

FIG. 11 illustrates power density of various solvent/water fractions for THC fuel cells in accordance with an embodiment.

FIG. 12 illustrates various ion exchange membrane power density curves for THC fuel cells in accordance with an embodiment.

FIG. 13 illustrates polarization curves and power density curves of various anode materials for THC fuel cells in accordance with an embodiment.

FIG. 14 illustrates an LC-MS chromatogram showing THC, p-THCQ/o-THCQ yield after 20 minutes at constant potential in accordance with an embodiment.

FIG. 15 illustrates current output with a bias potential of 0 V vs Ag/Ag⁺ with 2 μM THC and 0 M THC in a THC fuel cell in accordance with an embodiment.

FIG. 16A illustrates real-time chronoamperometry of a THC fuel cell in accordance with an embodiment.

FIG. 16B illustrates the correlation of an integration of total charge or measurement of maximum current from a THC fuel cell with various THC concentrations in accordance with an embodiment.

FIG. 17 illustrates comparison of cell potential and power density of THC fuel cell stack and THC H-cell performances in accordance with an embodiment.

### DETAILED DESCRIPTION

Turning now to the drawings, systems and methods for detecting phenolic cannabinoids using fuel cells are described. Many embodiments implement fuel cells to electrochemically detect phenolic cannabinoids by oxidizing the phenolic cannabinoids. Phenolic cannabinol can be oxidized to their corresponding quinones. Several embodiments implement a direct oxidation process of THC to detect the number of electrons during the oxidation for detection. Examples of phenolic cannabinoids include (but are not limited to) tetrahydrocannabinol (THC or Δ⁹-THC), Δ⁸-THC, cannabinol (CBN), and cannabidiol (CBD).

In some embodiments, THC oxidation can be a chemical process. In several embodiments, THC oxidation in the fuel cells can be an electrochemical process. Many embodiments implement THC including (but not limited to) in gas phase and/or solution phase in the oxidation process for detection. Many embodiments provide cannabinoid fuel cells that can detect THC in real time. In several embodiments, a higher THC input into the cannabinoid fuel cells can generate a higher measurable signal including (but not limited to) current, power, current density, power density, and total charge, corresponding to the THC oxidation processes.

Various types of cannabinoid fuel cells that can oxidize THC and detect the total charge of the oxidation processes are described. In many embodiments, the fuel cells include at least one cathode, at least one anode, at least one electrolyte (catholyte and/or anolyte), at least one ion exchange membrane, and at least one power source. In many embodiments, cathodes can comprise any catalyst materials including (but not limited to) transition metals, alloys, alloys comprising at least one transition metal element. Cathodes can include pure forms of the catalyst materials. In some embodiments, cathodes can include the catalyst materials supported on at least one support material including (but not limited to) carbon, fullerene, graphene, graphite, nanotubes, and carbon nanotubes. Examples of cathode used in an electrochemical platform include (but are not limited to): platinum on carbon cloth, platinum on carbon paper, and platinum/ruthenium on carbon cloth. In several embodiments, anodes can comprise any catalyst materials including (but not limited to) transition metals, metals, metal oxides, alloys, alloys comprising at least one transition metal element. Anodes can include pure forms of the catalyst materials. In a number of embodiments, anodes can include the catalyst materials supported on at least one support material including (but not limited to) carbon, carbon powder, carbon black, carbon black powder, fullerene, graphene, graphite, nanotubes, and carbon nanotubes. Examples of anode used in an electrochemical platform to oxidize THC include (but are not limited to): glassy carbon, platinum nanocrystals on glassy carbon, copper oxide (CuO), CuO modified with multi-wall carbon nanotube (MWCNT), Ni(OH)₂, Ni(OH)₂ modified with MWCNT, transition metals (such as, ruthenium (Ru), copper (Cu), palladium (Pd), platinum (Pt), iron (Fe), rhodium (Rh), nickel (Ni)), transition metals on carbon (such as, Ru on carbon (Ru/C), copper on carbon (Cu/C), palladium on carbon (Pd/C), platinum on carbon (Pt/C), iron on carbon (Fe/C), rhodium on carbon (Rh/C), nickel on carbon (Ni/C)), carbon black XC-72 (such as Vulcan XC72, Vulcan XC72R, both referred as Vulcan), Ru on Vulcan, Pt on Vulcan, Cu on Vulcan, Pd on Vulcan, Fe on Vulcan, Rh on Vulcan, Ni on Vulcan, Super P® carbon black powder, Cu on Super P®, Ni(OH)₂ on Super P®, and alloy combinations such as platinum-nickel on carbon (PtNi/C). As can readily be appreciated, any of a variety of cathode and/or anode material can be utilized as appropriate to the requirements of specific applications in accordance with various embodiments of the invention. Several embodiments implement platinum on carbon cloth cathode and a Ru/C anode for THC oxidation. Some embodiments implement a carbon cloth cathode and a Ni(OH)₂ modified with MWCNT anode for THC oxidation. Certain embodiments implement a carbon cloth cathode and a CuO modified with MWCNT anode for THC oxidation. In certain embodiments, a carbon cloth cathode and a Ru on Vulcan anode are implemented for THC oxidation. Some embodiments implement a carbon cloth cathode and a Pt on Vulcan anode for THC oxidation.

In several embodiments, electrolyte salts can be dissolved in solvents to function as catholyte and/or anolyte for cannabinoid fuel cells. In some embodiments, THC is soluble in anolytes and/or catholytes. Examples of electrolyte salt in cannabinoid fuel cells include (but are not limited to): NBu₄PF₆, NEt₄PF₆, LiPF₆, LiPF₄, NBu₄BF₄, NEt₄BF₄, NBu₄ClO₄, LiClO₄, and any combinations thereof. As can readily be appreciated, any of a variety of electrolyte salt can be utilized as appropriate to the requirements of specific applications in accordance with various embodiments of the invention. Certain embodiments use organic solvents including (but not limited to) acetonitrile as a solvent for the electrolyte salts. As can readily be appreciated, any of a variety of solvent can be utilized as appropriate to the requirements of specific applications in accordance with various embodiments of the invention.

Many embodiments provide optimum electrolyte solvent/water fractions and/or salt concentrations for cannabinoid fuel cells. In several embodiments, electrolyte salt concentration can range from about 0.01 M to about 0.5 M. In certain embodiments, the electrolyte solvent/water fraction can range from about 95% to about 100%. As can readily be appreciated, any of a variety of solvent/water fraction and salt concentration can be utilized as appropriate to the requirements of specific applications in accordance with various embodiments of the invention.

Various types of ion exchange membranes can be used in cannabinoid fuel cells. In many embodiments, ion exchange membranes that can conduct ions and/or protons can be used in cannabinoid fuel cells. Several embodiments utilize proton exchange membranes (PEM) in the fuel cells. Examples of ion exchange membranes can include (but are not limited to) Nafion® 117, Nafion® 112, Nafion® 212, Xion® PEM, Fumasep® F930, Fumasep® FKB-PK-130, Fumasep® F950, Fumasep® FS950, Fumasep® FKE-50, and anion Fumasep® FAS-30 for cannabinoid fuel cells. As can readily be appreciated, any of a variety of ion exchange membrane and/or proton exchange membrane can be utilized as appropriate to the requirements of specific applications in accordance with various embodiments of the invention.

Many embodiments eliminate the use of individual catholyte and/or anolyte in the fuel cells. In several embodiment, cathodes and/or anodes can be combined with membranes to form membrane electrode assemblies (MEA's). Cathodes and/or anodes can be in direct contact with the ion exchange membrane. Gas including (but not limited to) oxygen flow can be supplied to the cathodes. Ion exchange membrane may be hydrated to keep ions flowing.

The cannabinoid fuel cells in accordance with a number of embodiments can detect THC concentration of less than or equal to about 1 mM; or from about 1 μM to about 1 mM; or less than or equal to about 1 μM. During detection, a baseline signal of the fuel cell with no analyte added can be first recorded. The addition of THC to the cannabinoid fuel cells can generate a current peak that is higher than the baseline signal. An integration of the current peak can generate a total charge of the THC signal. Some embodiments provide that THC signals can have a linear relationship of the input THC concentration.

Systems and methods for cannabinoid fuel cells in accordance with various embodiments of the invention are discussed further below.

Phenolic Cannabinoids Detection with Fuel Cells

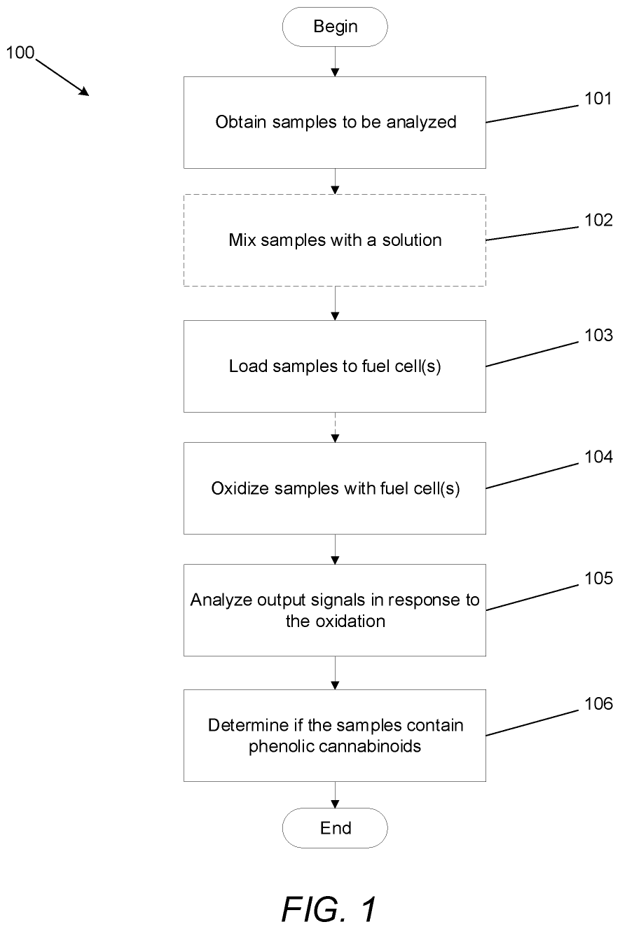

Many embodiments provide fuel cells that can perform oxidation processes including (but not limited to) chemical oxidation and/or electrochemical oxidation to directly oxidize phenolic cannabinoids including (but not limited to) tetrahydrocannabinol (THC or Δ⁹-THC), Δ⁸-THC, cannabinol (CBN), and cannabidiol (CBD) in solution phase and/or in gas phase for phenolic cannabinoids detection. A method for phenolic cannabinoids detection in a fuel cell in accordance with an embodiment of the invention is illustrated in FIG. 1. The process 100 can begin by obtaining a sample to be analyzed 101. Some embodiments include solution samples including (but not limited to) biofluids, tear, saliva, mucus, urine, sweat, blood, plasma. In some embodiments, a sample is in gas phase. Gas phase samples can be obtained from (but not limited to) breath. In some embodiments, a biological sample extracted from an individual can be used. In some embodiments, samples are put into solution or further diluted in a liquid. In some embodiments, samples are partially processed (e.g., centrifugation, filtration, etc.). In some embodiments, samples can be used as extracted from the source. As can readily be appreciated, any of a variety of solution samples can be utilized as appropriate to the requirements of specific applications in accordance with various embodiments of the invention.

Samples may be prepared by mixing with a solution 102. In many embodiments, the sample can be dissolved in a solvent including (but not limited to) aqueous solvent and/or organic solvent. Examples of solvent include (but are not limited to) acetonitrile. As can readily be appreciated, any of a variety of mixing solution can be utilized as appropriate to the requirements of specific applications. In a number of embodiments, samples can be loaded onto fuel cells directly and may not be mixed with a solution.

In a number of embodiments, the samples and/or mixed solutions can be loaded to the fuel cell to be oxidized 103. In many embodiments, fuel cells can directly oxidize phenolic cannabinoids for detection 104. The total charge transfer during the oxidation process can be measured. In certain embodiments, the oxidation process includes oxidizing THC to THCQ. THC oxidation processes in fuel cells in accordance with some embodiments can be carried out under ambient conditions such as at room temperature between about 20° C. and about 25° C. In certain embodiments, elevated temperatures may be used to improve fuel cell performances. In many embodiments, fuel cells can include at least one cathode, at least one anode, at least one ion exchange membrane, at least one electrolyte, and at least one power supply. Examples of cathode used in the fuel cell include (but are not limited to): platinum on carbon cloth, platinum on carbon paper, or platinum/ruthenium on carbon cloth. Examples of anode used in the fuel cell to oxidize THC include (but are not limited to): glassy carbon, platinum nanocrystals on glassy carbon, CuO, CuO modified with MWCNT, Ni(OH)₂, Ni(OH)₂ modified with MWCNT, transition metals, Ru, Ru/C, Cu/C, Pd/C, Pt/C, Fe/C, Rh/C, Ni/C, PtNi/C, Super P® carbon black powder, Cu on Super P®, and Ni(OH)₂ on Super P®. In some embodiments, the carbon substrate for the anode can be Vulcan XC72 or Vulcan XC72R (both can be referred as Vulcan), such as Ru/Vulcan, Cu/Vulcan, Pd/Vulcan, Pt/Vulcan, Fe/Vulcan, Rh/Vulcan, and Ni/Vulcan. As can readily be appreciated, any of a variety of cathode and/or anode material can be utilized as appropriate to the requirements of specific applications in accordance with various embodiments of the invention. The fuel cells can have catholyte for cathode and anolyte for anode. The catholyte and anolyte can use the same or different electrolyte salts and/or solvents. Examples of electrolyte salt include (but are not limited to): NBu₄PF₆, NEt₄PF₆, LiPF₆, LiPF₄, NBu₄BF₄, NEt₄BF₄, NBu₄ClO₄, LiClO₄, and any combinations thereof. Examples of solvent include (but are not limited to) acetonitrile. As can readily be appreciated, any of a variety of electrolyte can be utilized as appropriate to the requirements of specific applications in accordance with various embodiments of the invention. A current and/or a voltage signal can be applied to the fuel cells to initiate oxidation process.

In many embodiments, signals from the fuel cell can be directly measured as output 105. Several embodiments provide direct readout of the fuel cell performances including (but not limited to) current, power, and total charge as a result of the oxidation processes. Several embodiments can identify if oxidation of phenolic cannabinoids has taken place based on the signatures in total charge, current density and/or power density measurements. In a number of embodiments, the fuel cells provide real-time readout signals.

Based on the analysis results, samples can be identified if they contain phenolic cannabinoids or not 106. As oxidation of phenolic cannabinoids may generate unique signatures in fuel cell output signals, phenolic cannabinoids can be identified by the presence of such signatures. The fuel cell measurements collected by the analysis step can be processed in real-time in accordance with several embodiments. In several embodiments, a relationship between the readout signals and the concentration of phenolic cannabinoids can be established using samples with known phenolic cannabinoids concentration. Such relationship can be used to translate readout signals such as current, voltage, and/or power from fuel cells to phenolic cannabinoids concentration, such that the concentration of phenolic cannabinoids can be determined.

While various processes of cannabinoid fuel cells are described above with reference to FIG. 1, any of a process that includes various steps of the process can be performed in different orders and that certain steps may be optional according to some embodiments of the invention. As such, it should be clear that the various steps of the process could be used as appropriate to the requirements of specific applications. Furthermore, any of a variety of processes for detecting phenolic cannabinoids with a fuel cell appropriate to the requirements of a given application can be utilized in accordance with various embodiments of the invention. Processes for oxidizing THC with fuel cell breathalyzers in accordance with various embodiments of the invention are discussed further below.

Tetrahydrocannabinol Oxidation with Fuel Cells

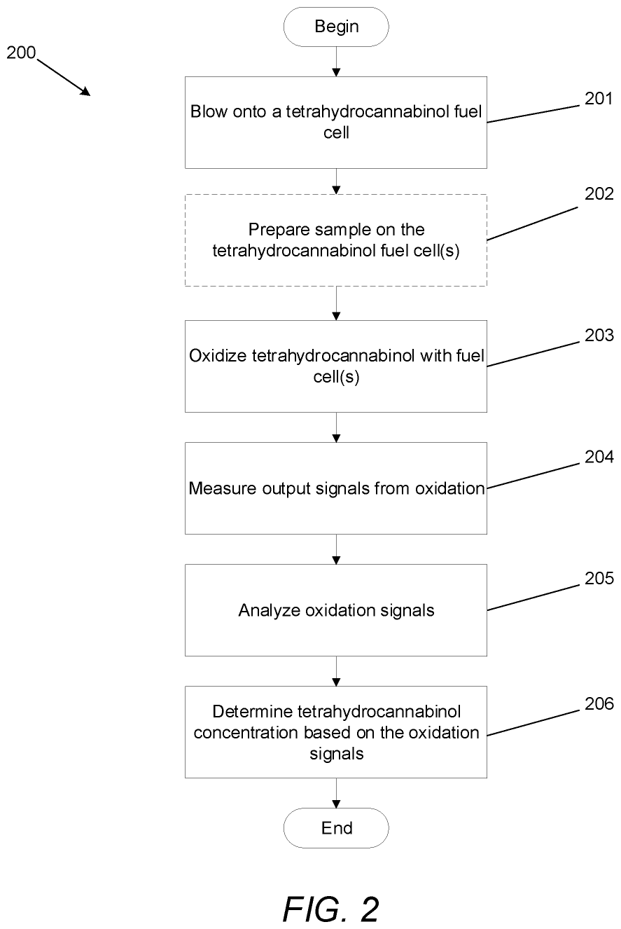

Many embodiments provide fuel cells including (but not limited to) a H-cells, fuel cell stacks, and flow cells that are able to oxidize THC in gas phase to corresponding oxidized products for detection. The cannabinoid fuel cells in accordance with some embodiments can be integrated in breathalyzers. In many embodiments, THC detection can be carried out with a multimodal breathalyzer and/or a dual modal alcohol marijuana breathalyzer. A method for detecting THC with a fuel cell in accordance with an embodiment of the invention is illustrated in FIG. 2. The process 200 can begin by obtaining a sample to be analyzed 201. In some embodiments, a sample is in gas phase. Gas phase samples can be obtained from (but not limited to) breath. In some embodiments, an individual can exhale into a collection device including (but not limited to) a breathalyzer for a certain time period. In various embodiments, pressure regulators can be attached to regulate the pressure of the breath into the fuel cells. Within the sample collection device can be an analytic unit configured to electrochemically oxidize THC. As can readily be appreciated, any of a variety of methods to obtain gas phase samples for a breathalyzer can be utilized as appropriate to the requirements of specific applications in accordance with various embodiments of the invention.

Samples can be prepared by mixing with an electrolyte 202. In many embodiments, the sample can be dissolved in a solvent including (but not limited to) aqueous solvent and/or organic solvent. In some embodiments, the sample may not be dissolved in a solvent to be detected by the fuel cell. Certain embodiments can use organic solvent including (but not limited to) acetonitrile as a solvent. As can readily be appreciated, any of a variety of solvent can be utilized as appropriate to the requirements of specific applications. In many embodiments, samples in gas phase can be directly applied to an electrolyte.

In a number of embodiments, the samples and/or the prepared samples can be oxidized electrochemically with the fuel cells 203. In many embodiments, fuel cells for oxidizing THC includes at least one cathode, at least one anode, at least one ion exchange membrane, at least one chamber, at least one electrolyte, and at least one power source. Examples of cathodes used in the fuel cell include (but are not limited to): platinum on carbon cloth, platinum on carbon paper, or platinum/ruthenium on carbon cloth. Examples of anodes used in the fuel cell to oxidize THC include (but are not limited to): glassy carbon, Pt nanocrystals on glassy carbon, CuO, CuO modified with MWCNT, Ni(OH)₂, Ni(OH)₂ modified with MWCNT, transition metals, transition metals with carbon support (such as, Cu/C, Pd/C, Pt/C, Fe/C, Rh/C, Ni/C, Ru/C), PtNi/C, Super P® carbon black powder, Cu on Super P®, and Ni(OH)₂ on Super P®. In some embodiments, the carbon substrate for the anode can be Vulcan XC72 or Vulcan XC72R (both are referred as Vulcan), such as Ru/Vulcan, Cu/Vulcan, Pd/Vulcan, Pt/Vulcan, Fe/Vulcan, Rh/Vulcan, and Ni/Vulcan. As can readily be appreciated, any of a variety of cathode and/or anode materials can be utilized as appropriate to the requirements of specific applications in accordance with various embodiments of the invention. The fuel cells can have catholyte for cathode and anolyte for anode. The catholyte and anolyte can use the same or different electrolyte salts and/or solvents. Examples of electrolyte salt include (but are not limited to): NBu₄PF₆, NEt₄PF₆, LiPF₆, LiPF₄, NBu₄BF₄, NEt₄BF₄, NBu₄ClO₄, LiClO₄, and any combinations thereof. Examples of solvent include (but are not limited to) acetonitrile. As can readily be appreciated, any of a variety of electrolyte salt and/or solvent can be utilized as appropriate to the requirements of specific applications in accordance with various embodiments of the invention.

Electrolyte can be placed in various ways in the breathalyzer including (but not limited to) in a container, in a flow channel, in a fluid channel, on a substrate, and/or incorporated in a hydrogel in accordance with several embodiments. A current or voltage can be applied to the breathalyzer to initiate oxidation process. Electrochemical oxidation process of THC in the fuel cell in accordance with some embodiments can be carried out under ambient conditions such as at room temperature. Certain embodiments operate the fuel cell between around 20° C. to around 25° C. Elevated temperatures from about 30° C. to about 40° C. may improve fuel cell performances.

In several embodiments, the fuel cells can generate output signals in response to the oxidation processes 204. Oxidation of THC can be analyzed directly and in real-time in accordance with certain embodiments. Several embodiment provide THC oxidation can have signatures in output signals such as current and/or power. In some embodiments, output signals from the THC oxidation can be analyzed 205. The analysis can include (but are not limited to) removing background noise, enhancing signal to noise ratio, deconvoluting THC oxidation signals. The oxidation of THC can be identified based on the signatures in total charge, current density and/or power density measurements.

In several embodiments, concentration of THC can be determined by the analyzed output signals in real time 206. In some embodiments, a relationship between the readout signals and the concentration of THC can be established using samples with known THC concentration. In certain embodiments, the THC concentration has a linear relationship with the readout signals. Such relationship can be used to determiner THC concentrations in real time based on readout signals such as current, voltage, and/or power.

While various processes of detecting THC in a sample with fuel cell breathalyzers are described above with reference to FIG. 2, any of a process that includes various steps of the process can be performed in different orders and that certain steps may be optional according to some embodiments of the invention. As such, it should be clear that the various steps of the process could be used as appropriate to the requirements of specific applications. Furthermore, any of a variety of processes for detecting THC with a fuel cell appropriate to the requirements of a given application can be utilized in accordance with various embodiments of the invention.

### Tetrahydrocannabinol

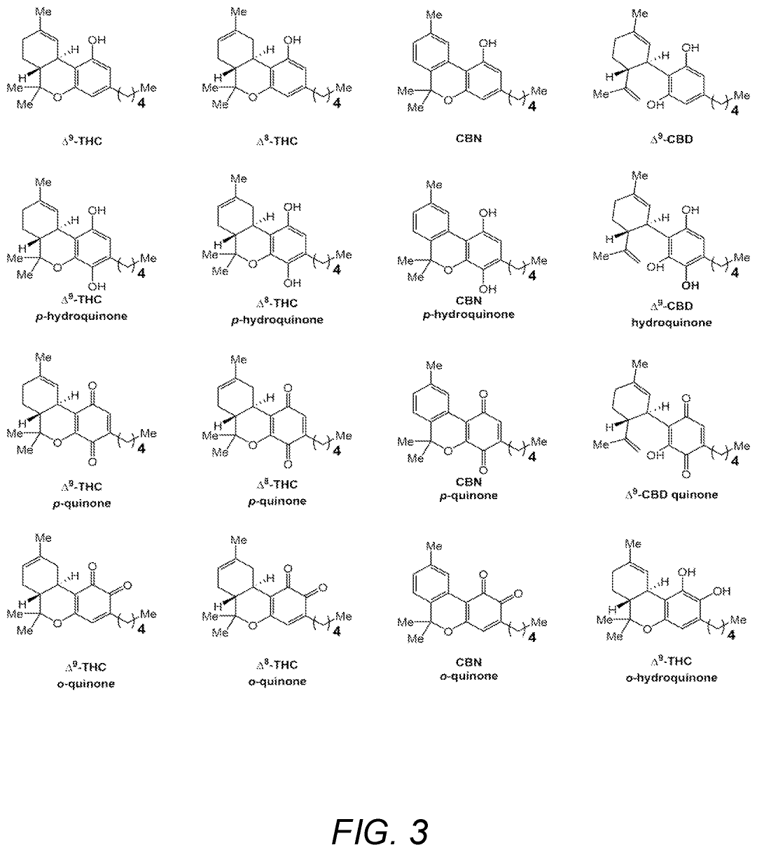

Δ⁹-Tetrahydrocannabinol (Δ⁹-THC or THC) is one of at least 113 cannabinoids identified in cannabis. THC may be the primary psychoactive constituent of cannabis. With chemical name (−)-trans-Δ⁹-tetrahydrocannabinol, THC can refer to cannabinoid isomers. In many embodiments, THC and Δ⁹-THC can be used exchangably to refer to tetrahydrocannabinol. In several embodiments, THCQ and Δ⁹-THCQ can be used exchangably to refer to tetrahydrocannabinol p-quinone and/or quinoidal isomer o-quinone. FIG. 3 illustrates the chemical structure of different phenolic cannabinoids. FIG. 3 includes chemical structures of Δ⁹-THC, Δ⁸-THC, CBD, and CBN.

The legalization and decriminalization of marijuana and related cannabinoids have become more common. Clinical trials show impairment can negatively impact ability to operate machinery. However, current testing and detection technologies that rely on blood, urine, or saliva do not always correlate to impairment. (See, e.g., J. Röhrich, et al., J. Anal. Toxicol., 2010, 34, 196-203; M. Divagar, et al., IEEE Sens. J. 2021, 21, 22758-22766; M. Dagar, et al., Talanta, 2022, 238, 123048; the disclosures of which are incorporated herein by references.) This may be because impairment can be most pronounced within 3-4 hours of usage, whereas THC can persist in bodily fluids for time periods as long as several weeks. (See, e.g., M. DeGregorio, et al., Sci. Rep. 2021, 11, 22776; A. G. Verstraete, Ther. Drug Monit. 2004, 26, 200-205; the disclosures of which are incorporated herein by references.) Few options are available for rapid detection that correlate with the window of impairment. As such, there exists a need for a fair forensic tool capable of detecting marijuana in the short window of impairment.

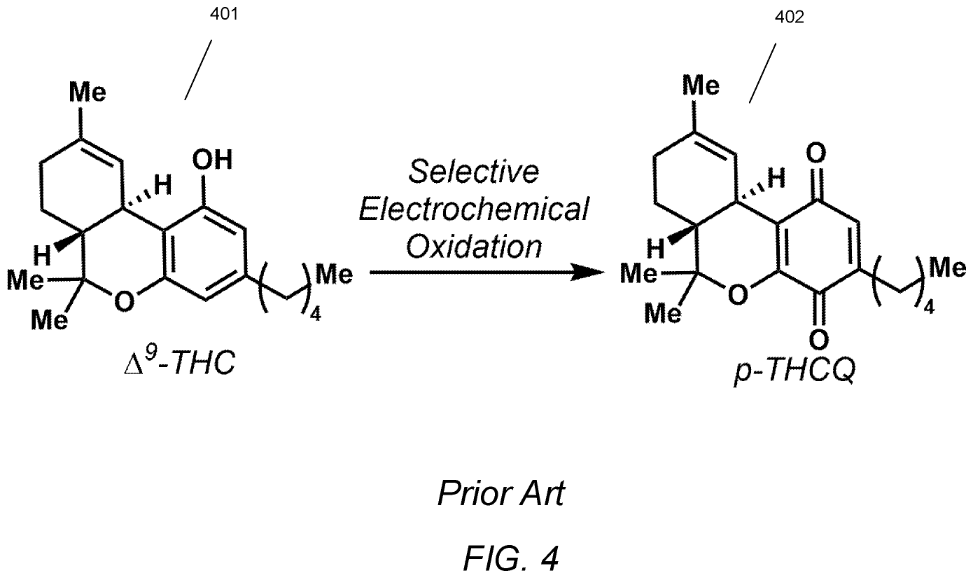

Breath analysis can be a promising avenue based on recent clinical trials, although breath-based detection technologies are currently limited. Promising approaches include the use of fluorescence, chemiresistors, and mass spectrometry. (See, e.g., U.S. Pat. No. 9,921,234 B1 to M. S. Lynn, et al.; S. I. Hwang, et al., ACS Sens. 2019, 4, 2084-2093; PCT Publication No. WO 2018/200794 A1 to A. Star, et al.; PCT Publication No. WO 2017/147687 A2 to R. Attariwala, et al.; M. T. Costanzo, et al., Int. J. Mass Spectrom. 2017, 422, 188-196; H. Lai, et al., Anal. Bioanal. Chem. 2008, 392, 105-113; the disclosures of which are incorporated herein by references.) A promising and ideal approach involves the use of electrochemistry. (See, e.g., PCT Publication No. WO 2020/167828 A1 to B. M. Dweik; U.S. Patent Publication No. 2020/0025740 A1 to B. M. Dweik, et al.; U.S. Patent Publication No. 2020/0124625 A1 to T. Dunlop, et al.; the disclosures of which are incorporated herein by references.) Darzi and Garg have previously reported the chemical and/or electrochemical oxidation of THC to THCQ. FIG. 4 illustrates reaction scheme of THC oxidation to THCQ. (See, e.g., PCT Publication No. WO 2021/087453 A1 to N. K. Garg et al.; the disclosure of which is incorporated herein by reference in its entirety.)

Many embodiments implement fuel cells including (but not limited to) H-Cells, fuel cell stacks, and flow cells to oxidize phenolic cannabinoids including (but not limited to) Δ⁹-tetrahydrocannabinol. The cannabinoid fuel cells can be used in marijuana breathalyzers. Several embodiments implement current-producing H-Cells that rely on the oxidation of Δ⁹-tetrahydrocannabinol. Some embodiments provide optimized conditions including (but not limited to) anode materials, membrane materials, solvents, electrolytes, and concentrations, for the phenolic cannabinoids detecting fuel cells. The current and power densities could improve at least 4-fold and 5-fold, respectively, using the optimized conditions.

Many embodiments provide the detection of phenolic cannabinoids by oxidizing phenolic cannabinoids using fuel cells including (but not limited to) H-cells, stack fuel cells, and flow cells. The detection of Δ⁹-THC can be achieved by oxidizing Δ⁹-THC using fuel cells. Δ⁹-THC can be oxidized to corresponding p-quinone and/or o-quinone, Δ⁹-THCQ. A reaction scheme of THC oxidizing to THCQ is illustrated in FIG. 4. 401 illustrates THC in its chemical structure. 402 illustrates THCQ in its chemical structure. THCQ can be p-THCQ and/or o-THCQ. The oxidation of Δ⁹-THC can be achieved chemically and/or electrochemically. Many embodiments provide integration of Δ⁹-THC fuel cells into multimodal marijuana breathalyzer devices. Processes for detecting THC using fuel cells in accordance with various embodiments of the invention are discussed further below.

### Cannabinoid Fuel Cells for THC Oxidation

Fuel cell technology has been revolutionary in many fields and provides the basis for many alcohol breathalyzers. Few examples of fuel cells for phenol oxidation have been reported, particularly in the context of wastewater remediation. (See, e.g., G. S. Buzzo, et al., Catal. Commun. 2015, 59, 113-115; H. M. Zhang, et al., Sep. Purif. Technol. 2017, 172, 152-157; R. Wu, et al., J. Am. Chem. Soc. 2022, 144, 1556-1571; S. Liu, et al., NANO, 2019, 10, 1950134; Y. Wu, et al., RSC Adv., 2020, 10, 39447-39454; A. Ziaedini, et al., Fuel Cells 2018, 4, 526-534; the disclosures of which are incorporated herein by references.)

Many embodiments implement cannabinoid fuel cells that can oxidize THC. Cannabinoid fuel cells can be inexpensive, mass producible, and useful in a host of applications including (but not limited to) dual THC-alcohol breathalyzers and generating electricity from hemp waste in accordance with several embodiments. The fuel cells can be in various constructs including (but not limited to) H-cells, fuel cell stacks, and flow cells for THC detection. Cannabinoid fuel cells in accordance with some embodiments can be made with various materials including (but not limited to) plastics, metals, alloys, ceramics, glasses, non-reactive materials, papers, textiles, and any combinations thereof. A number of embodiments provide that the fuel cells can be fabricated using various methods including (but not limited to) molding, casting, glass blowing, additive manufacturing, printing, and any combinations thereof. In certain embodiments, the fuel cells can be purchased as ready-to-use products. The cannabinoid fuel cells can oxidize THC. In many embodiments, THC in both solution phase and gas phase can be detected using cannabinoid fuel cells. In some embodiments, THC oxidation with fuel cells use mild reaction conditions. Certain embodiments provide that THC can be directly oxidized to form THCQ. In many embodiments, cannabinoid fuel cells can spontaneously oxidize THC and generate a current signal for detection. The cannabinoid fuel cells provide real-time readout of THC oxidation. In a number of embodiments, THC fuel cells use a constant current; or constant current and a catalyst to oxidize THC. In several embodiments, background noise can be corrected in order to retrieve signals of oxidized THC products. The noise correction system and/or the signal analysis system can be part of the cannabinoid fuel cells; or can be attached to the cannabinoid fuel cells as attachments.

Many embodiments provide cannabinoid fuel cells can include at least one cathode, at least one anode, at least one chamber, at least one ion exchange membrane, and at least one power supply. In several embodiments, THC oxidation reactions can take place at the anodes. Examples of anode materials include (but are not limited to) glassy carbon, Pt nanocrystals on glassy carbon, CuO, CuO modified with MWCNT, Ni(OH)₂, Ni(OH)₂ modified with MWCNT, transition metals, and transition metals with carbon support. In some embodiments, the carbon substrate for the anode can be carbon cloth, carbon powder, carbon black powder, Super P®, carbon coated substrate, Vulcan XC72 or Vulcan XC72R. The anodes of cannabinoid fuel cells have at least one dimension in the scale from nanometer to micrometer to millimeter. THC oxidation can generate THCQ. Reduction reactions of molecules including (but not limited to) oxygen at the cathodes can balance the charge flow. Examples of cathode materials include (but are not limited to) platinum, platinum on carbon, platinum on carbon cloth, platinum/ruthenium, platinum/ruthenium on carbon, and platinum/ruthenium on carbon cloth. Some embodiments provide that the at least one chamber may include solvents and/or electrolytes. In certain embodiments, the at least one chamber may not need solvents and/or electrolytes. The anode chamber in accordance with certain embodiments can include at least one electrolyte and at least one solvent that THC in either liquid form or gas form can be soluble in. Examples of solvent include (but are not limited to) acetonitrile. Examples of electrolyte salts include (but are not limited to) NBu₄BF₄, and NEt₄PF₆. Ion exchange membranes can connect to at least one anode chamber and to at least one cathode chamber and facilitate ion including (but not limited to) proton flow. Examples of ion exchange membranes include (but are not limited to) Nafion® 117, Nafion® 112, Fumasep® F930, and Fumasep® F950. An outside lead can be established to complete the electron flow pathway.

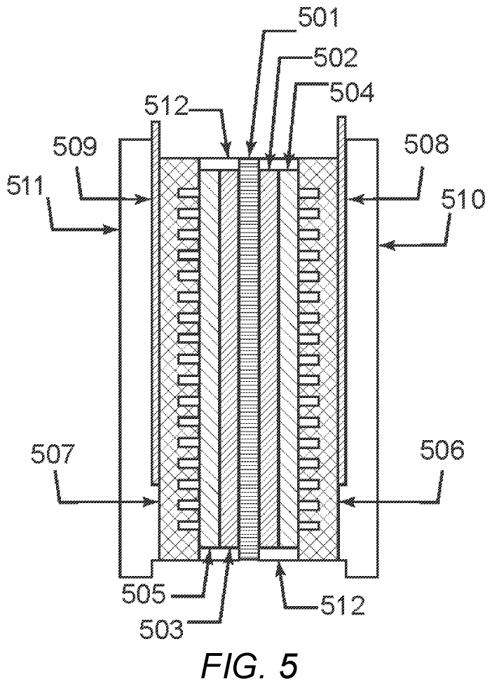

A cannabinoid fuel cell stack in accordance with an embodiment of the invention is illustrated in FIG. 5. The cannabinoid fuel cell stack includes an ion exchange membrane 501 such as a proton exchange membrane sandwiched by a cathode 502 and an anode 503. The cathode 502 is in contact with a gas diffusion layer 504, and the anode is in contact with a gas diffusion layer 505. The gas diffusion layer 504 and 505 can be made of the same materials or different materials. The gas diffusion layer 504 on the cathode side is in contact with a cathode flow plate 506. The gas diffusion layer 505 on the anode side is in contact with an anode flow plate 507. The cathode flow plate 506 is connected with a cathode current collector 508. The anode flow plate 507 is connected with an anode current collector 509. A cathode end plate 510 completes the cathode side of the fuel cell stack. An anode end plate 511 completes the anode side of the fuel cell stack. Teflon gasket material 512 can be used to seal the fuel cell stack. THC oxidation reaction can occur on the anode. A counter reduction reaction can occur on the cathode.

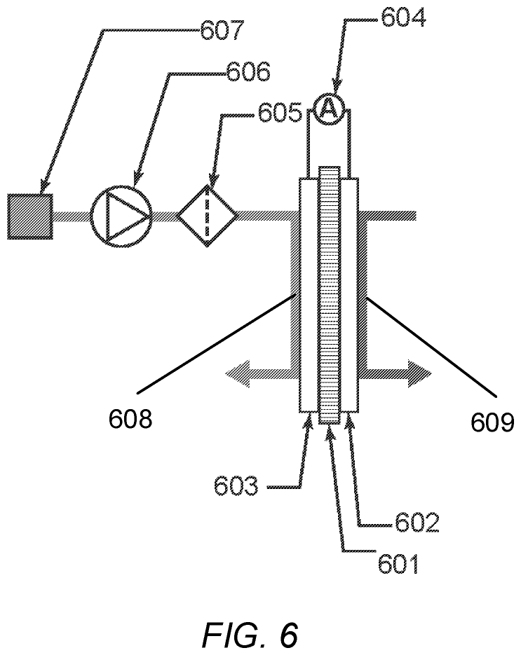

A cannabinoid fuel cell in accordance with an embodiment of the invention is illustrated in FIG. 6. The cannabinoid fuel cell comprises an ion exchange membrane 601, a cathode 602, an anode 603, a micro controller 604, an in-line filter holder 605, a liquid pump 506, and an electrolyte reservoir 607. The ion exchange membrane 601 such as a proton exchange membrane is sandwiched between the cathode 602 and the anode 603. The ion exchange membrane can facilitate ion flow, allowing for the generation of current (flow of electrons). The micro controller 604 senses the voltage signal or the current signal. The electrolyte reservoir 607 supplies electrolyte to the fuel cell via the liquid pump 606. The electrolyte reservoir 607 and the liquid pump 606 may be optional if electrolyte is not used in the fuel cell. 608 shows liquid anolyte flow path, including electrolyte flow and the introduction of THC into the electrolyte by passing through the filter 605 such as a THC-laden filter. At the anode 603, direct oxidation of THC can give rise to THCQ for detection. A counter reduction of O₂ to H₂O may occur at the cathode 602. 609 shows passive and/or air flow path.

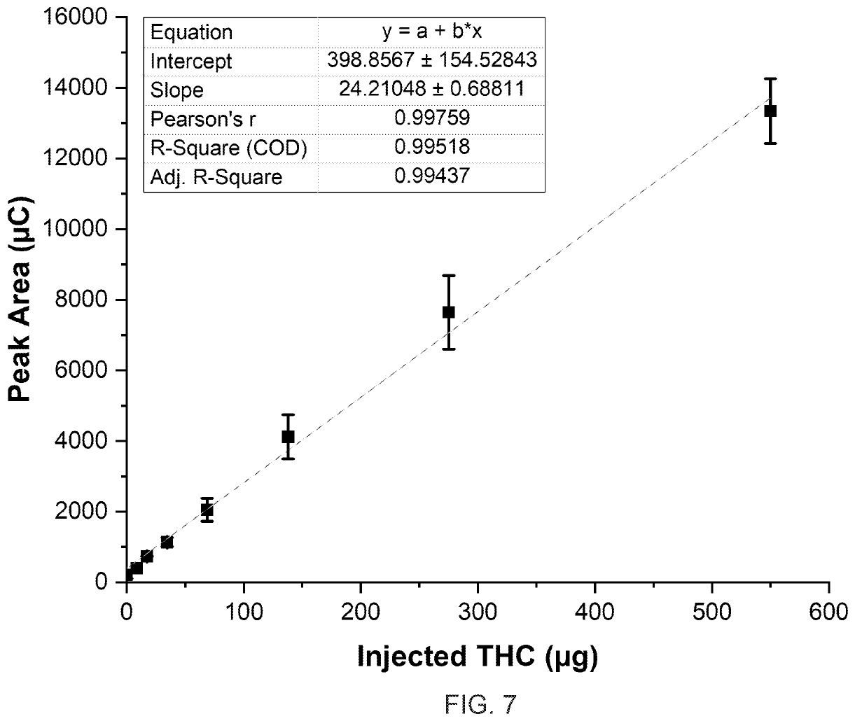

In many embodiments, the output of the cannabinoid fuel cells represents a direct measurement of the input cannabinoid concentration. In several embodiments, the cannabinoid fuel cells produce a linear response to the amount of cannabinoid fuel (such as, THC). FIG. 7 illustrates a cannabinoid fuel cell output signals at different THC concentrations in accordance with an embodiment of the invention. FIG. 7 shows the peak area of a cannabinoid fuel cell, such as a fuel cell stack, at THC (cannabinoid fuel) concentration from about 0 μg to about 600 μg. The peak area can be calculated by integrating the peak current at the respective THC concentration with time. The squares are the averaged peak area response for each fuel amount (two measurements are averaged), and the error bars represent standard error. The fitted line shows a linear fit, and the inset text shows the statistics for the linear fit.

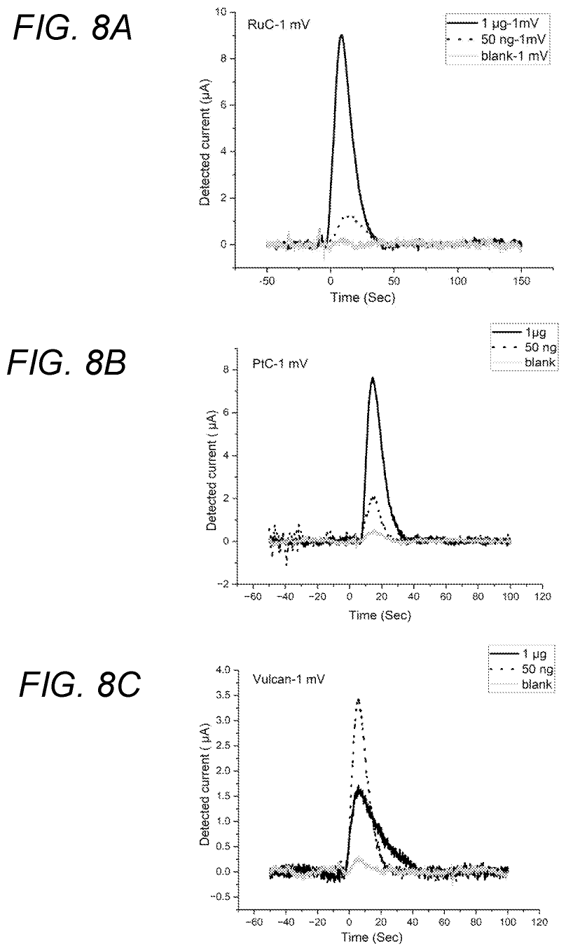

Anode materials can affect cannabinoid fuel cell performance. Various catalyst materials can be integrated into anodes to enhance the fuel cell performance. Different types of catalysts can be combined to further improve the fuel cell performance. Some embodiments implement economical catalysts such as carbon, carbon black, graphene, as catalysts for the cannabinoid fuel cells. Several embodiments combine the economical catalysts with metal catalysts (such as, Ru, Pt, Pd, Ni) to improve conversion efficiency. FIGS. 8A-8C illustrate the effect of various anode materials on fuel cell performance in accordance with an embodiment. FIG. 8A shows Ru on Vulcan as the anode material for the fuel cell. FIG. 8B shows Pt on Vulcan as the anode material for the fuel cell. FIG. 8C shows Vulcan as the anode material for the fuel cell. THC concentration at about 0 ng, at about 50 ng, and at about 1000 ng are injected to the fuel cell and the responding current signals (current peak height and peak area) are measured.

Table 1 summarizes the fuel cell performance at different THC concentrations. All results in Table 1 are an average of 4 measurements. As can be seen, the hybrid anode materials Ru/Vulcan and Pt/Vulcan have better performance than the Vulcan anode material.

TABLE 1
Effect of anode material on fuel cell performance.

| | Bias | THC | Peak | Peak |
|---|---|---|---|---|
| Anode | Potential | Injection | Height | Area |
| Material | (mV) | (ng) | (mA) | (mC) |
| Ru/Vulcan | 1 mV | 0 | 0.275 | 1.56 |
| | | 50 | 1.00 | 12.10 |
| | | 1000 | 1.70 | 38.70 |
| Pt/Vulcan | 1 mV | 0 | 1.73 | 12.80 |
| | | 50 | 2.07 | 17.40 |
| | | 1000 | 132 | 43.20 |
| Vulcan | 1 mV | 0 | 0.419 | 6.50 |
| | | 50 | 1.40 | 16.00 |
| | | 1000 | 1.83 | 22.00 |

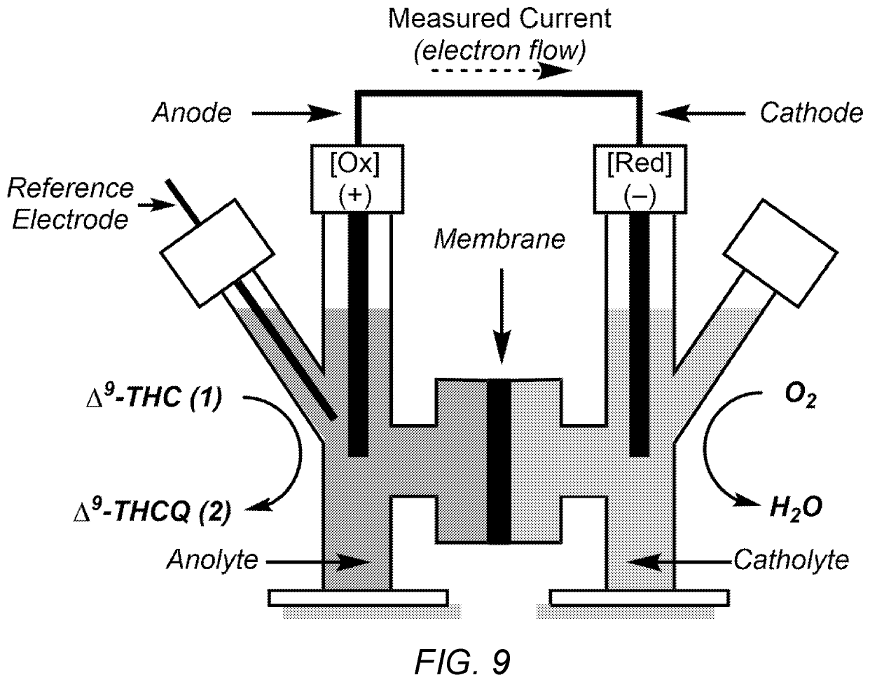

Many embodiments use H-cells as cannabinoid fuel cells. An H-cell type fuel cell for cannabinoid detection in accordance with an embodiment of the invention is illustrated in FIG. 9. The cannabinoid H-cells can electrochemically oxidize THC using a catalyst and/or constant current and generate a current signal through the oxidation of THC (1). The cannabinoid H-cell can be made with glass or any non-reactive materials. FIG. 9 shows that the H-cell can have two half cells with each half cell having a capacity of less than about 10 mL; or greater than about 10 mL. The two half cells can be connected with an ion exchange membrane. The anode half-cell includes anolyte, and the cathode half-cell includes catholyte. The anode and the reference electrode are immersed in the anolyte. The cathode is immersed in the catholyte. The half cells, each equipped with a sealing electrode port and a reference electrode port, can be connected using a flange and a membrane holder.

Many embodiments provide reaction conditions for cannabinoid H-cells including (but not limited to) THC concentrations, electrolytes, electrolyte concentrations, electrolyte solvent fraction and salt concentrations, membrane materials, cathode materials, and anode materials. Several embodiments provide optimized conditions for higher open circuit potential and/or power density of the cannabinoid fuel cells.

In many embodiments, background signal can often be observed in the absence of THC. In order to improve the net signal, a normalized THC signal to the background noise can be used to show fuel cell performances. In some embodiments, background noise can be measured with THC H-cells in the absence of THC. Background noise can include background power density and/or background current density. In certain embodiments, power density signal-to-noise ratio (SNR) can be calculated using the following equation:

Power Density SNR=THC power density÷background power density (1)

Power density SNR can be calculated based on Eq. 1 unless otherwise specified. In some embodiments, current density SNR can be calculated using the following equation:

Current Density SNR=THC current density÷background current density (2)

Current density SNR are calculated based on Eq. 2 unless otherwise specified. Some embodiments provide relative power density signal-to-noise ratio and/or relative current density signal-to-noise ratio (SNRᵣₑₗ) to compare device performances. Certain embodiments with pristine conditions provide a power density signal-to-noise ratio (SNR) of about 0.844 and current density signal-to-noise of about 0.845 can be used to normalize power density SNRᵣₑₗ and current density SNRᵣₑₗ, respectively. Power density SNRᵣₑₗ can be calculated using the following equation:

$$Power density {SNR}_{rel} = \frac{( THC power density \div background power density )}{0.844} \qquad ( 3 )$$

Power density SNRᵣₑₗ are calculated based on Eq. 3 unless otherwise specified. Current density SNRᵣₑₗ can be calculated using the following equation:

$$Current density {SNR}_{rel} = \frac{( THC current density \div background current density )}{0.845} \qquad ( 4 )$$

Relative current density SNRᵣₑₗ are calculated based on Eq. 4 unless otherwise specified.

While various systems and processes of detecting THC with fuel cells are described above with reference to FIG. 5 through FIG. 9, any of a fuel cell system that includes various elements for cannabinoid detection can be performed according to some embodiments of the invention. As such, it should be clear that the various elements could be used as appropriate to the requirements of specific applications. Furthermore, any of a variety of elements for THC fuel cells appropriate to the requirements of a given application can be utilized in accordance with various embodiments of the invention.

### Cannabinoid Fuel Cell Optimization

Many embodiments provide reaction conditions for cannabinoid H-cells including (but not limited to) THC concentrations, electrolytes, electrolyte concentrations, electrolyte solvent fraction and salt concentrations, membrane materials, cathode materials, and anode materials. Various cathode materials can be used in cannabinoid fuel cells. Some embodiments screen cathode materials including (but not limited to) platinum on carbon cloth, platinum on carbon paper, and platinum/ruthenium on carbon cloth. In certain embodiments, at least 5 cycles of cyclic voltammetry can be performed to check the consistency of the cathodes relative to Fc/Fc⁺. Cathode materials with good consistency are chosen to continue the screening tests. Reaction conditions H-cell tests in accordance with several embodiments include: platinum on carbon cloth, platinum on carbon paper, or platinum/ruthenium on carbon cloth as the cathode connected working electrode, glassy carbon disc electrode as the anode connected counter electrode, Ag/AgNO₃ and 0.1 M LiClO₄ in acetonitrile as the reference electrode, 0.1 M LiClO₄ in acetonitrile as the catholyte, and about 5 mg THC and 0.1 M LiClO₄ in acetonitrile as the anolyte. Table 2 summarizes the effect of cathode materials on fuel cell performance. Table 2 lists highest power density and current density SNR of H-cell with various cathode materials. Cloth platinum on carbon shows a highest power density of about 0.157 mW/cm² and a current density SNR of about 0.16 mA/cm².

TABLE 2
Highest power density and current density SNR of H-cell with
cloth platinum on carbon, paper platinum on carbon, and
cloth platinum/ruthenium on carbon as cathode materials.

| | Highest Power | Current Density |
|---|---|---|
| Cathode Materials | Density (mW/cm²) | SNR (mA/cm²) |
| Pt/C cloth | 0.04026 | 0.845 |
| 4 mg/cm² | | |
| PtRu/C cloth | 0.05055 | 0.794 |
| 4 mg/cm² | | |
| Pt/C paper | 0.2491 | 1.064 |
| 4 mg/cm² | | |

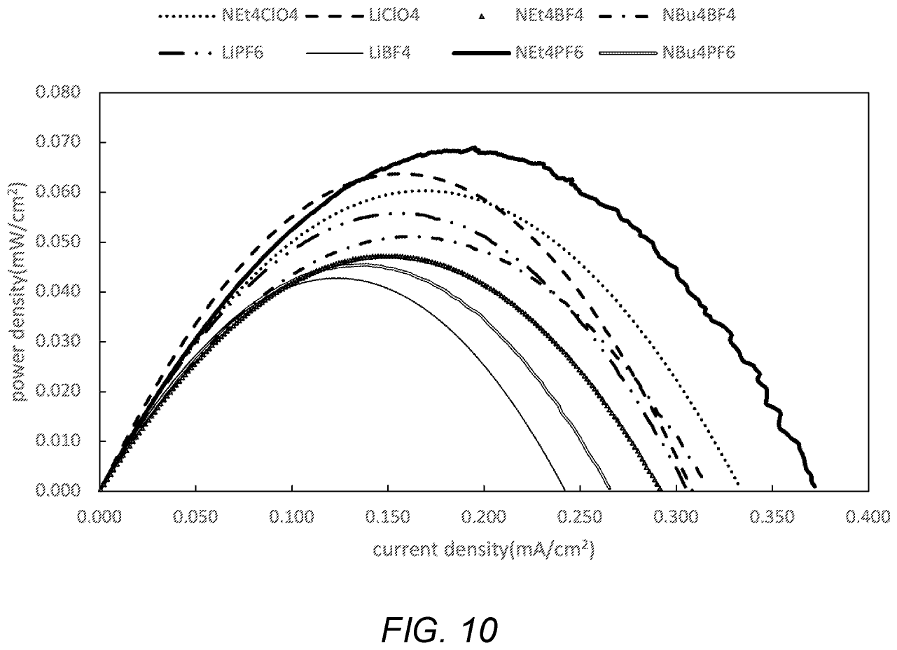

Various electrolyte including (but not limited to) catholyte and anolyte for cannabinoid fuel cells. Several embodiments use power density tests to screen suitable electrolyte salts for catholyte and anolyte of cannabinoid H-cells. The test conditions include 4 mg/cm² Pt black on about 5 cm² carbon felt as the cathode, glassy carbon working electrode as the anode, Ag/AgNO₃ and 0.1 M LiClO₄ in acetonitrile as the reference electrode. Catholyte in accordance with certain embodiments can include catholyte salt including (but not limited to) NBu₄PF₆, NEt₄PF₆, LiPF₆, LiPF₄, NBu₄BF₄, NEt₄BF₄, NBu₄ClO₄, LiClO₄, and any combinations thereof, dissolved in acetonitrile. Catholyte salt concentration can be about 0.1 M. Oxygen gas can be sparged in the catholyte during the test. Anolyte in accordance with certain embodiments can include THC, anolyte salt including (but not limited to) NBu₄PF₆, NEt₄PF₆, LiPF₆, LiPF₄, NBu₄BF₄, NEt₄BF₄, NBu₄ClO₄, LiClO₄, and any combinations thereof, dissolved in acetonitrile. THC can be about 5 mg, and anolyte salt can be about 0.1 M. Nitrogen gas can be flown to the anolyte during the test. Catholyte salt and anolyte salt for cannabinoid fuel cells can be the same or different. FIG. 10 illustrates power density curve of THC H-cells with different electrolyte salts in accordance with an embodiment. The power density curves of THC H-cells in each one of the electrolyte salts: NBu₄PF₆, NEt₄PF₆, LiPF₆, LiPF₄, NBu₄BF₄, NEt₄BF₄, NEt₄ClO₄, LiClO₄, are shown in FIG. 10.

Table 3 summarizes cell potential and power density of cannabinoid H-cells with various electrolyte salts. Table 3 lists power density and current density SNR of THC H-cells with various electrolytes in acetonitrile as catholyte and anolyte. NEt₄PF₆ electrolyte salt gives a highest power density of about 0.069 mW/cm² for THC H-cells. NEt₄PF₆, LiPF₆, and NBu₄BF₄ can result in a high current density SNR for THC H-cells.

TABLE 3
Power density and current density SNR of
THC H-cells with various electrolytes.

| | Power Density | Current Density |
|---|---|---|
| Electrolyte Salt | (mW/cm²) | SNR (mA/cm²) |
| NBu₄PF₆ | 0.0455 | 0.804 |
| NEt₄PF₆ | 0.0690 | 1.07 |
| LiBF₄ | 0.0427 | 0.920 |
| LiPF₆ | 0.0506 | 1.00 |
| NBu₄BF₄ | 0.0512 | 0.794 |
| NEt₄BF₄ | 0.0472 | 0.705 |
| LiClO₄ | 0.0637 | 0.745 |
| NEt₄ClO₄ | 0.0603 | 0.905 |

Table 4 lists performances of THC H-cells with various electrolytes. The test conditions include glassy carbon as the anode, 4 mg/cm² platinum carbon cloth as cathode, Ag/AgNO₃ and 0.1 M LiClO₄ in acetonitrile as the reference electrode, Nafion® 117 as the proton exchange membrane, and about 5 mg THC (2.27 mM). Performance of THC H-cells include open circuit potential (OCP), power density, current density, power density SNR, current density SNR, relative power density SNR, and relative current density SNR. Electrolyte salts include about 0.1 M of NBu₄PF₆, NEt₄PF₆, LiPF₆, LiPF₄, NBu₄BF₄, NEt₄BF₄, NEt₄ClO₄, and LiClO₄.

TABLE 4
Performance of THC H-cells with various electrolytes.

| | | | Power | Current | | | | |
|---|---|---|---|---|---|---|---|---|
| | Electro- | | Density | Density | Power | Current | Power | Current |
| | lyte | OCP | (mW/ | (mA/ | Density | Density | Density | Density |
| Cathode | (conc.) | (mV) | cm²) | cm²) | SNR | SNR | SNRᵣₑₗ | SNRᵣₑₗ |
| Pt/C | NBu₄PF₆ | 556 | 0.000046 | 0.00023 | 3.19 | 3.20 | 3.79 | 3.79 |
| (3 mm² | | | | | | | | |
| disc) | | | | | | | | |
| Pt/C | NBu₄BF₄ | 649 | 0.00005 | 0.000014 | 2.17 | 2.16 | 2.57 | 2.56 |
| (3 mm² | (0.1M) | | | | | | | |
| disc) | | | | | | | | |
| 4 mg/cm² | NBu₄BF₄ | 520 | 0.0403 | 0.125 | 0.844 | 0.845 | 1.00 | 1.00 |
| Pt/C | (0.1M) | | | | | | | |
| 4 mg/cm² | NEt₄BF₄ | 608 | 0.0472 | 0.149 | 0.708 | 0.705 | 0.839 | 0.834 |
| Pt/C | (0.1M) | | | | | | | |
| 4 mg/cm² | LIBF₄ | 672 | 0.0427 | 0.125 | 0.918 | 0.920 | 1.09 | 1.09 |
| Pt/C | (0.1M) | | | | | | | |
| 4 mg/cm² | LiClO₄ | 786 | 0.0637 | 0.156 | 0.747 | 0.745 | 0.885 | 0.881 |
| Pt/C | (0.1M) | | | | | | | |
| 4 mg/cm² | NEt₄ClO₄ | 692 | 0.0603 | 0.168 | 0.908 | 0.905 | 1.08 | 1.07 |
| Pt/C | (0.1M) | | | | | | | |
| 4 mg/cm² | LiPF₆ | 689 | 0.0558 | 0.155 | 1.00 | 1.00 | 1.19 | 1.19 |
| Pt/C | (0.1M) | | | | | | | |
| 4 mg/cm² | NBu₄PF₆ | 650 | 0.0455 | 0.140 | 0.802 | 0.804 | 0.950 | 0.951 |
| Pt/C | (0.1M) | | | | | | | |
| 4 mg/cm² | NEt₄PF₆ | 608 | 0.0690 | 0.195 | 1.07 | 1.07 | 1.27 | 1.27 |
| Pt/C | (0.1M) | | | | | | | |

Various electrolyte solvent fraction and/or salt concentration can be used in cannabinoid fuel cells. Some embodiments provide power density tests to screen suitable electrolyte solvent fraction and/or salt concentration of cannabinoid H-cells. The test conditions include 4 mg/cm² Pt black on about 5 cm² carbon felt as the cathode, glassy carbon working electrode as the anode, Ag/AgNO₃ and 0.1 M NEt₄PF₆ in acetonitrile as the reference electrode and Nafion® 117 as the ion exchange membrane. Several embodiments provide catholyte can include various concentration of catholyte salt including (but not limited to) NEt₄PF₆ dissolved in acetonitrile. Oxygen gas can be flown to the catholyte during the test. Certain embodiments provide anolyte can include THC, various concentration of anolyte salt including (but not limited to) NEt₄PF₆, dissolved in acetonitrile. Nitrogen gas can be flown to the anolyte during the test. THC concentration can be about 5 mg. Table 5 lists solvent fractions and salt concentration variables for the screening tests.

TABLE 5
Solvent fraction and salt concentration variables.

| Salt | Solvent Fraction | | | | |
|---|---|---|---|---|---|
| Concentration | 100% | 99% | 98% | 97% | 96% |
| 0.5M | ✓ | ✓ | ✓ | ✓ | ✓ |
| 0.25M | ✓ | ✓ | ✓ | ✓ | ✓ |
| 0.1M | ✓ | ✓ | ✓ | ✓ | ✓ |
| 0.05M | ✓ | ✓ | ✓ | ✓ | ✓ |
| 0.01M | ✓ | ✓ | ✓ | ✓ | ✓ |

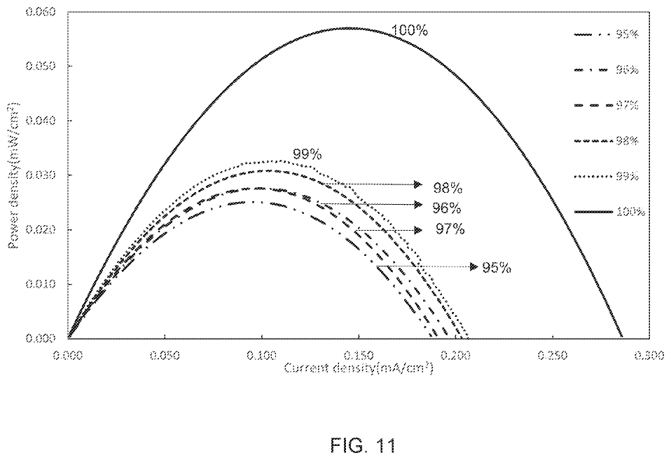

FIG. 11 illustrates power densities of THC H-cells with various water/acetonitrile fractions under about 0.05 M NEt₄PF₆ concentration in accordance with an embodiment. When THC H-cell have 100% acetonitrile (MeCN) as the electrolyte, the cells can obtain a highest power density than other MeCN/water fractions. Under the 100% MeCN condition, about 0.05 M NEt₄PF₆ can have a highest current density SNR. Table 6 lists comparison of power density of THC H-cells with variable solvent/water fractions and electrolyte concentrations. Table 7 lists power density of THC H-cell with different MeCN/water fractions and 0.05 M NEt₄PF₆.

TABLE 6
Comparison of power density of THC H-cell with variable
solvent fraction and salt concentration

| | Solvent Fraction | | | | | |
|---|---|---|---|---|---|---|
| | 100% | | 99% | 98% | 97% | 96% |
| NEt₄PF₆ | Power Density | Current Density | Power Density | | | |
| Concentration | (mW/cm²) | SNR (mW/cm²) | (mW/cm²) | | | |
| 0.5M | 0.175 | 0.813 | 0.149 | 0.127 | 0.0992 | 0.0957 |
| 0.25M | 0.128 | 0.981 | 0.102 | 0.0774 | 0.0723 | 0.0529 |
| 0.1M | 0.0689 | 1.07 | 0.0483 | 0.0424 | 0.0400 | 0.0260 |
| 0.05M | 0.0452 | 1.14 | 0.0338 | 0.0233 | 0.0276 | 0.0163 |
| 0.01M | 0.0138 | 1.03 | 0.0083 | 0.0060 | 0.0066 | 0.0036 |

TABLE 7
Power density of THC H-cell with variable MeCN fractions

| | MeCN Fraction | | | | | |
|---|---|---|---|---|---|---|
| NEt₄PF₆ | 100% | 99% | 98% | 97% | 96% | 95% |
| Concentration | Power Density (mW/cm²) | | | | | |
| 0.05M | 0.0452 | 0.0327 | 0.0308 | 0.0276 | 0.0276 | 0.0251 |

Table 8 lists performances of THC H-cells with various electrolyte salt concentrations in 100% acetonitrile. The test conditions include glassy carbon as the anode, 4 mg/cm² Pt/C as cathode, Ag/AgNO₃ and 0.1 M NEt₄PF₆ in acetonitrile as the reference electrode, Nafion® 117 as the proton exchange membrane, and about 5 mg THC (2.27 mM). Performance of THC H-cells include open circuit potential (OCP), power density, current density, power density SNR, current density SNR, relative power density SNRᵣₑₗ, and relative current density SNRᵣₑₗ. Electrolyte salt of NEt₄PF₆ concentration ranges from about 0.01 M to 0.5 M.

TABLE 8
Performance of THC H-cells
with various electrolyte salt concentrations.

| | | | Cur- | | | | |
|---|---|---|---|---|---|---|---|
| | | Power | rent | | Cur- | | Cur- |
| | | Den- | Den- | Power | rent | Power | rent |
| Electro- | | sity | sity | Den- | Den- | Den- | Den- |
| lyte | OCP | (mW/ | (mA/ | sity | sity | sity | sity |
| (conc.) | (mV) | cm²) | cm²) | SNR | SNR | SNRᵣₑₗ | SNRᵣₑₗ |
| NEt₄PF₆ | 709 | 0.0138 | 0.0385 | 1.01 | 1.03 | 1.20 | 1.23 |
| (0.01M) + | | | | | | | |
| 100% | | | | | | | |
| MeCN | | | | | | | |
| NEt₄PF₆ | 674 | 0.0452 | 0.131 | 1.11 | 1.14 | 1.31 | 1.34 |
| (0.05M) + | | | | | | | |
| 100% | | | | | | | |
| MeCN | | | | | | | |
| NEt₄PF₆ | 608 | 0.0689 | 0.195 | 1.07 | 1.07 | 1.27 | 1.27 |
| (0.1M) + | | | | | | | |
| 100% | | | | | | | |
| MeCN | | | | | | | |
| NEt₄PF₆ | 653 | 0.128 | 0.372 | 0.982 | 0.981 | 1.16 | 1.16 |
| (0.25M) + | | | | | | | |
| 100% | | | | | | | |
| MeCN | | | | | | | |
| NEt₄PF₆ | 637 | 0.175 | 0.510 | 0.812 | 0.813 | 0.962 | 0.962 |
| (0.5M) + | | | | | | | |
| 100% | | | | | | | |
| MeCN | | | | | | | |

Various ion exchange membranes can be used in cannabinoid fuel cells. Some embodiments provide power density tests to screen suitable ion exchange membranes of cannabinoid H-cells. The test conditions include 4 mg/cm² Pt black on about 5 cm² carbon felt as the cathode, glassy carbon working electrode as the anode, Ag/AgNO₃ and 0.1 M NEt₄PF₆ in acetonitrile as the reference electrode, 0.05 M NEt₄PF₆ in acetonitrile as catholyte, and 5 mg THC and 0.05 M NEt₄PF₆ in acetonitrile as anolyte. Proton exchange membranes can include (but are not limited to) Nafion® 117, Nafion® 112, Nafion® 212, Xion® PEM, Fumasep® F930, Fumasep® FKB-PK-130, Fumasep® F950, Fumasep® FS950, Fumasep® FKE-50, and anion Fumasep® FAS-30. Several embodiments provide galvanostatic EIS tests for membrane resistance tests. Parameters for the tests include, Initial frequency of about 10⁶ Hz, final frequency of about 0.1 Hz, DC current of about 1.5 E-5 A, AC current of about 1.5 E-5 A, points/decay of about 10.

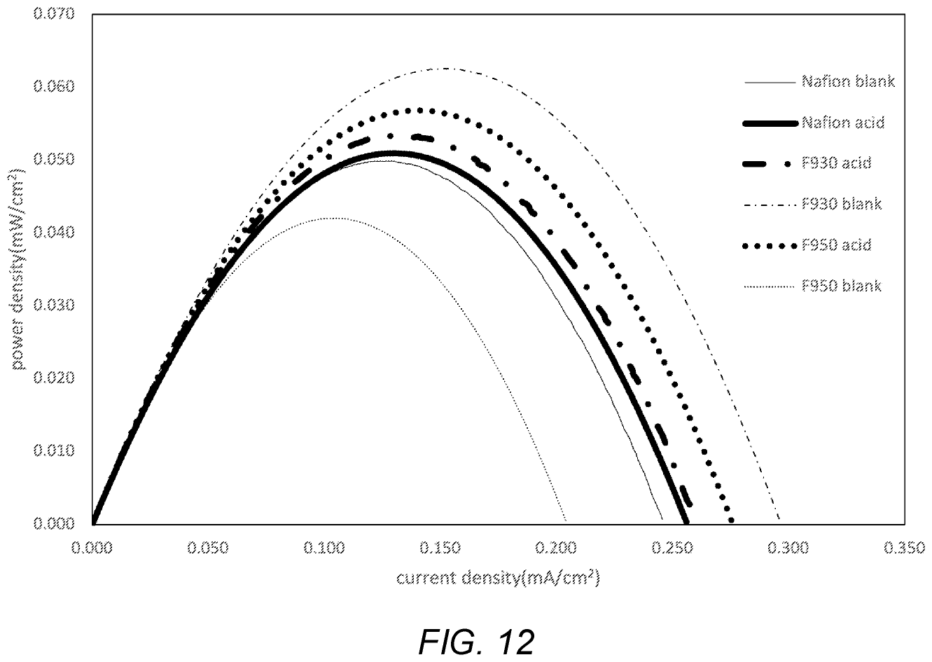

FIG. 12 illustrates power density curves of THC H-cells with different membranes pretreated in H₂SO₄ in accordance with an embodiment. The power density curves of each of the membranes: Nafion® 117 without acid treatment (marked as Nafion blank), Nafion® 117 pretreated with H₂SO₄ (marked as Nafion acid), Fumasep® F930 without acid treatment (marked as F930 blank), Fumasep® F930 treated with H₂SO₄ (marked as F930 acid), Fumasep® F950 without acid treatment (marked as F950 blank), and Fumasep® F950 treated with H₂SO₄ (marked as F950 acid), are shown in FIG. 12. Table 9 lists performances of THC H-cells with various ion exchange membranes.

TABLE 9
THC H-cells with various ion exchange membranes

| | Power | EIS-Membrane | EIS-Bulk | |
|---|---|---|---|---|
| | Density | Resistance | Resistance | Current |
| Membrane | (mW/cm2) | (ohm) | (ohm) | Density SNR |
| Nafion ® 117 | 0.0452 | 13.6 | 539 | 1.14 |
| Fumasep ® | 0.0551 | 1.21 | 450 | 1.05 |
| FS-990-PK | | | | |
| Fumasep ® | 0.0521 | 24.2 | 484 | 1.03 |
| FS-9100-PK | | | | |
| Fumasep ® | 0.0700 | 8.87 | 494 | 1.03 |
| FS-950 | | | | |
| Fumasep ® | 0.0585 | 19.8 | 359 | 0.093 |
| F930 | | | | |
| Fumasep ® | 0.0501 | 5.63 | 492 | 1.20 |
| F950 | | | | |
| Nafion ® 212 | 0.000003 | Out of range | Out of range | NA |
| Xion ® PEM | NA | Out of range | Out of range | NA |
| Fumasep ® | 0.000011 | Out of range | Out of range | NA |
| FKE-50 | | | | |
| Fumasep ® | 0.00312 | 16.0 | 7076 | NA |
| FKB-PK-130 | | | | |
| Fumasep ® | 0.000007 | 6.17 | 530 | NA |
| FAS-30 | | | | |

Table 10 lists performances of THC H-cells with various ion exchange membranes. The test conditions include glassy carbon as the anode, 4 mg/cm² Pt/C as cathode, Ag/AgNO₃ and 0.1 M NEt₄PF₆ in acetonitrile as the reference electrode, 0.05 M NEt₄PF₆ in acetonitrile as catholyte, and 5 mg THC and 0.05 M NEt₄PF₆ in acetonitrile as anolyte. Performance of THC H-cells include open circuit potential (OCP), power density, current density, power density SNR, current density SNR, relative power density SNRᵣₑₗ, and relative current density SNRᵣₑₗ.

TABLE 10
THC H-cell performances with different ion exchange membranes.

| | | | Cur- | | | | |
|---|---|---|---|---|---|---|---|
| | | Power | rent | | Cur- | | Cur- |
| | | Den- | Den- | Power | rent | Power | rent |
| | | sity | sity | Den- | Den- | Den- | Den- |
| | OCP | (mW/ | (mW/ | sity | sity | sity | sity |
| Membrane | (mV) | cm²) | cm²) | SNR | SNR | SNRᵣₑₗ | SNRᵣₑₗ |
| Nafion ® | 674 | 0.0452 | 0.131 | 1.11 | 1.14 | 1.31 | 1.34 |
| 117 | | | | | | | |
| Fumasep ® | 760 | 0.0551 | 0.142 | 1.04 | 1.05 | 1.24 | 1.24 |
| FS-990- | | | | | | | |
| PK | | | | | | | |
| Fumasep ® | 740 | 0.0521 | 0.136 | 1.03 | 1.03 | 1.22 | 1.22 |
| FS-9100- | | | | | | | |
| PK | | | | | | | |
| Fumasep ® | 881 | 0.0701 | 0.155 | 1.03 | 1.03 | 1.22 | 1.21 |
| FS-950 | | | | | | | |
| Fumasep ® | 784 | 0.0585 | 0.143 | 0.926 | 0.925 | 1.10 | 1.09 |
| F930 | | | | | | | |
| Fumasep ® | 766 | 0.0502 | 0.126 | 1.20 | 1.20 | 1.42 | 1.42 |
| F950 | | | | | | | |

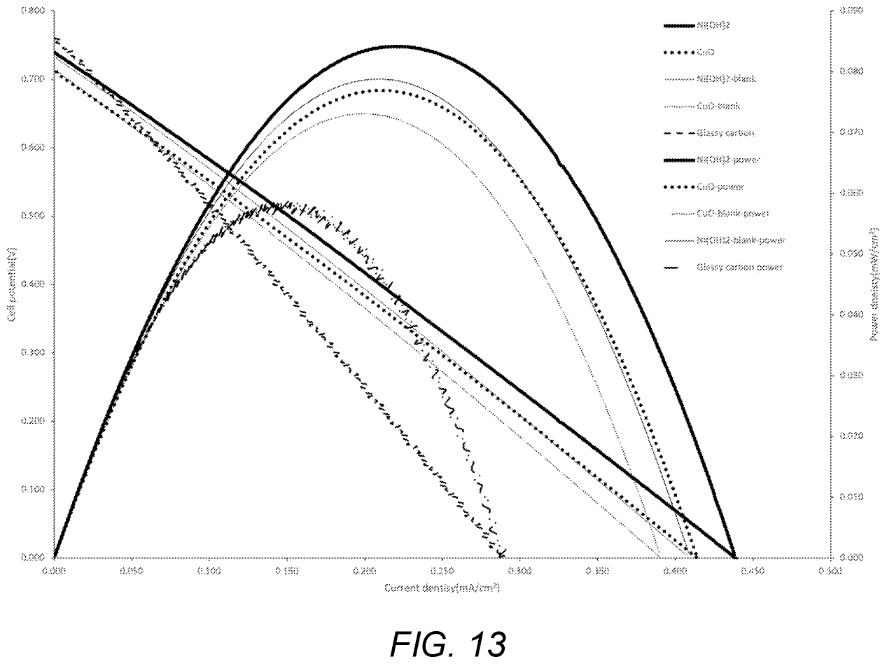

Various anode materials can be used in cannabinoid fuel cells. Some embodiments provide power density tests to screen suitable anode materials of cannabinoid H-cells. The test conditions include 4 mg/cm² Pt black on about 5 cm² carbon felt as the cathode, Ag/AgNO₃ and 0.1 M NEt₄PF₆ in acetonitrile as the reference electrode, Fumasep® F950 as ion exchange membrane 0.05 M NEt₄PF₆ in acetonitrile as catholyte, and 5 mg THC and 0.05 M NEt₄PF₆ in acetonitrile as anolyte. Anode materials include (but are not limited to) Ni(OH)₂/MWCNTs, CuO/MWCNTs, glassy carbon electrode, CuC, Pd/C, Pt/C, Fe/C, Pd/C, Rh/C, Ni/C, Ru/C, PtNi, Cu/SuperP, and Ni(OH)₂/SuperP. Anode materials can have various substrates including (but not limited to) MWCNT, C60, C70, and super P. Anode catalyst activity tests can be examined with cyclic voltammetry. Test conditions of cyclic voltammetry (CV) include, THC (5.00 mg, 2.27 mmol) and 0.1 M NBu₄PF₆ in acetonitrile as electrolyte, test anode material as the working electrode, Pt wire as the counter electrode, Ag/AgNO₃ in 0.1 M NBu₄PF₆ in acetonitrile as the reference electrode. Cyclic voltammograms of Ni(OH)₂/MWCNT as the working electrode shows a greater oxidative current response than CuO/MWCNT in the electrolyte with THC. When using CuO/MWCNT as the anode, the peak current response decreases with increasing scan cycles. Ni(OH)₂/MWCNT catalyst activity is stable after the third CV scan. FIG. 13 illustrates polarization curves and power density curves of THC H-cell with various anodes in accordance with an embodiment. Power density and cell potential curves of each of the anodes: Ni(OH)₂, Ni(OH)₂/MWCNT, CuO, CuO/MWCNT, and glassy carbon, are shown in FIG. 13. Table 11 lists power density and current density SNR of THC H-cell with various anode materials.

TABLE 11
THC H-cell performances with various anode catalysts.

| | Power Density | Current |
|---|---|---|
| Anode Catalyst | (mW/cm²) | Density SNR |
| Ni(OH)₂/MWCNT | 0.0964 | 1.11 |
| CuO/MWCNT | 0.0814 | 1.17 |
| Glassy carbon | 0.0502 | 1.20 |

Table 12 lists performances of THC H-cells with various anode materials and anode materials with various substrates. The test conditions include 4 mg/cm² Pt black on 5 cm² carbon felt as the cathode, Ag/AgNO₃ and 0.1 M NEt₄PF₆ in acetonitrile as the reference electrode, Fumasep® F950 as ion exchange membrane, 0.05 M NEt₄PF₆ in acetonitrile as catholyte, and 5 mg THC and 0.05 M NEt₄PF₆ in acetonitrile as anolyte. Performance of THC H-cells include open circuit potential (OCP), power density, current density, power density SNR, current density SNR, relative power density SNRᵣₑₗ, and relative current density SNRᵣₑₗ.

TABLE 12
THC H-cell performances with different anode materials.

| | | | | Power | Current | Power | Current | Power | Current |
|---|---|---|---|---|---|---|---|---|---|
| | | | OCP | Density | Density | Density | Density | Density | Density |
| | Anode | Membrane | (mV) | (mW/cm²) | (mW/cm²) | SNR | SNR | SNRᵣₑₗ | SNRᵣₑₗ |
| Anode | CuO/ | Nafion ® | 1092 | 0.102 | 0.559 | 1.11 | 1.00 | 1.32 | 1.19 |
| Materials | MWCNT | 117 | | | | | | | |
| | CuO/ | Fumasep ® | 888 | 0.0814 | 0.0695 | 1.26 | 1.17 | 1.49 | 1.39 |
| | MWCNT | F950 | | | | | | | |
| | Ni(OH)₂/ | Nafion ® | 1111 | 0.113 | 0.578 | 1.00 | 1.00 | 1.19 | 1.18 |
| | MWCNT | 117 | | | | | | | |
| | Ni(OH)₂/ | Fumasep ® | 1014 | 0.0964 | 0.188 | 1.11 | 1.11 | 1.32 | 1.32 |
| | MWCNT | F950 | | | | | | | |
| | CuC | Fumasep ® | 886 | 0.0759 | 0.167 | 0.865 | 0.865 | 0.844 | 1.02 |
| | | F950 | | | | | | | |
| | Pt/C | Fumasep ® | 907 | 0.0774 | 0.167 | 1.03 | 1.03 | 1.22 | 1.21 |
| | | F950 | | | | | | | |
| | Fe/C | Fumasep ® | 931 | 0.0874 | 0.186 | 0.988 | 0.988 | 1.17 | 1.17 |
| | | F950 | | | | | | | |
| | Pd/C | Fumasep ® | 896 | 0.0755 | 0.163 | 1.04 | 1.04 | 1.24 | 1.23 |
| | | F950 | | | | | | | |
| | Rh/C | Fumasep ® | 896 | 0.0779 | 0.169 | 1.05 | 1.05 | 1.24 | 1.24 |
| | | F950 | | | | | | | |
| | Ni/C | Fumasep ® | 883 | 0.0780 | 0.173 | 1.03 | 1.03 | 1.22 | 1.22 |
| | | F950 | | | | | | | |
| | Ru/C | Nafion ® | 1021 | 0.0876 | 0.512 | 1.11 | 1.00 | 1.31 | 1.18 |
| | | 117 | | | | | | | |
| | Ru/C | Fumasep ® | 923 | 0.0784 | 0.164 | 1.20 | 1.20 | 1.42 | 1.42 |
| | | F950 | | | | | | | |
| Anode | MWCNT | Fumasep ® | 1032 | 0.0929 | 0.173 | 1.14 | 1.14 | 1.36 | 1.35 |
| Materials | | F950 | | | | | | | |
| with | C60 | Fumasep ® | 672 | 0.0368 | 0.108 | 0.897 | 0.894 | 1.06 | 1.06 |
| various | | F950 | | | | | | | |
| substrate | C70 | Fumasep ® | 699 | 0.0433 | 0.120 | 0.820 | 0.820 | 0.971 | 0.970 |
| | | F950 | | | | | | | |
| | Super | Fumasep ® | 654 | 0.0451 | 0.134 | 1.16 | 1.16 | 1.37 | 1.37 |
| | P ® | F950 | | | | | | | |
| Anode | Cu(I)/ | Fumasep ® | 946 | 0.0910 | 0.187 | 1.10 | 1.10 | 1.31 | 1.30 |
| Materials | Super P ® | F950 | | | | | | | |
| | Ni(OH)₂/ | Fumasep ® | 999 | 0.0793 | 0.154 | 0.861 | 0.862 | 1.02 | 1.02 |
| | Super P ® | F950 | | | | | | | |
| | PtNi | Fumasep ® | 860 | 0.0606 | 0.137 | 1.13 | 1.13 | 1.34 | 1.33 |
| | | F950 | | | | | | | |

Various THC concentrations can be detected with cannabinoid fuel cells. Some embodiments provide power density tests to test THC concentration of cannabinoid H-cells. The test conditions include 4 mg/cm² Pt black on about 5 cm² carbon felt as the cathode, Ni(OH)₂/MWCNT as the anode, Ag/AgNO₃ and 0.1 M NEt₄PF₆ in acetonitrile as the reference electrode, Fumasep® F950 as ion exchange membrane, 0.05 M NEt₄PF₆ in acetonitrile as catholyte with oxygen bubbling, and various concentration of THC and M NEt₄PF₆ in acetonitrile as anolyte. THC concentrations can range from about mM to about 2.27 mM. Table 13 lists power density and current density SNR with various THC concentrations at about 0 mM THC, about 0.5 mg THC, about 1 mg THC, about 2.5 mg THC, and about 5 mg THC.

TABLE 13
THC H-cell power density and current density SNR with
various THC concentrations (optimal conditions)

| | Power Density | Current |
|---|---|---|
| THC Concentration | (mW/cm²V) | Density SNR |
| Blank (0 mM) | 0.0818 | 1.00 |
| 5 mg (2.27 mM) | 0.0856 | 1.05 |
| 2.5 mg (1.14 mM) | 0.0821 | 1.01 |
| 1 mg (0.450 mM) | 0.0872 | 1.07 |
| 0.5 mg (0.230 mM) | 0.0784 | 0.957 |

Many embodiments provide a list of reaction conditions shown in Table 14. Reaction conditions used in the oxidation of THC in the divided H-Cell include (except otherwise stated) about 5 μM THC dissolved in acetonitrile, a cation exchange membrane made of Nafion®117, and 4 mg/cm² Pt on 1 cm×5 cm carbon cloth cathode. For entry 1 of Table 14, a 3 mm disc electrodes for both the cathode (Pt nanocrystal on carbon) and anode (glassy carbon) are used. This can result in a measurable current, albeit with poor signal strength. The cathode material of Pt nanocrystal on carbon can have poor reproducibility. Entry 2 of Table 14 uses a commercially available 1 cm×5 cm⁴ mg/cm² Pt on carbon cloth electrode material. The change of cathode material leads to an increase in open circuit potential, current and power density (entry 1 vs. entry 2). The results from entry 2 can be used as a baseline for assessing fuel cell performance. In order to improve the overall signal over this baseline condition, a ratio of signal to background noise normalized to entry 2 can be reported as relative current/power signal-to-noise (SNRᵣₑₗ). Entries 3 and 4 provide fuel cell performances with a different electrolyte. NEt₄PF₆ shows improved performances than NBu₄BF₄. Further, by decreasing the electrolyte concentration (entry 5), the current and power densities SNRᵣₑₗ can increase to 2.65 and 1.22 respectively. Several embodiments provide fuel cell performances with different cation conducting membranes as found in entries 6-8. The use of Nafion® 212 membrane may lead to an inactive cell. The use of F930 and F950 may lead to improved current and power densities SNRᵣₑₗ. Some embodiments provide fuel cell performances with different anode materials. Several 3 mm disc electrode anode materials are provided (entries 9-11) with the optimized Fumapem® F950 membrane. Ru/C may have promising results (entry 11) and show relatively high power and current densities of about 0.0780 mW/cm² and about 0.164 mA/cm² respectively. This result represents an increase in power density of about 5 orders of magnitude in comparison to entry 1 while maintaining a relatively high ratio of signal to noise.

TABLE 14
Reaction conditions for cannabinoid fuel cells.

| | | | | Open | | | | |
|---|---|---|---|---|---|---|---|---|
| | | | | Circuit | Power | Current | Power | Current |
| | | Electrolyte | | Potential | Density | Density | Density | Density |
| Entry | Anode | (conc.) | Membrane | (V) | (mW/cm²) | (mW/cm²) | SNRᵣₑₗ | SNRᵣₑₗ |
| 1* | Glassy | NBu₄BF₄ | Nafion ® | 0.649 | 5.00 E−5 | 1.40 E−5 | 2.57 | 5.57 |
| | Carbon | (0.1M) | 117 | | | | | |
| 2 | Glassy | NBu₄BF₄ | Nafion ® | 0.520 | 0.040 | 0.125 | 1.00 | 1.00 |
| | Carbon | (0.1M) | 117 | | | | | |
| 3 | Glassy | NBu₄PF₆ | Nafion ® | 0.650 | 0.045 | 0.140 | 0.95 | 2.07 |
| | Carbon | (0.1M) | 117 | | | | | |
| 4 | Glassy | NEt₄PF₆ | Nafion ® | 0.608 | 0.069 | 0.195 | 1.20 | 2.60 |
| | Carbon | (0.1M) | 117 | | | | | |
| 5 | Glassy | NEt₄PF₆ | Nafion ® | 0.674 | 0.045 | 0.131 | 1.22 | 2.65 |
| | Carbon | (0.05M) | 117 | | | | | |
| 6 | Glassy | NEt₄PF₆ | Nafion ® | 0.183 | 3.0 E−6 | N/A | N/A | N/A |
| | Carbon | (0.05M) | 212 | | | | | |
| 7 | Glassy | NEt₄PF₆ | Fumasep ® | 0.784 | 0.053 | 0.134 | 1.01 | 2.19 |
| | Carbon | (0.05M) | F930 | | | | | |
| 8 | Glassy | NEt₄PF₆ | Fumasep ® | 0.766 | 0.050 | 0.126 | 1.42 | 3.09 |
| | Carbon | (0.05M) | F950 | | | | | |
| 9 | CuO/ | NEt₄PF₆ | Fumasep ® | 0.888 | 0.081 | 0.070 | 1.49 | 3.01 |
| | MWCNT | (0.05M) | F950 | | | | | |
| 10 | Ni(OH)₂/ | NEt₄PF₆ | Fumasep ® | 1.01 | 0.096 | 0.188 | 1.32 | 2.86 |
| | MWCNT | (0.05M) | F950 | | | | | |
| 11 | Ru/C | NEt₄PF₆ | Fumasep ® | 0.923 | 0.078 | 0.164 | 1.42 | 3.09 |
| | | (0.05M) | F950 | | | | | |

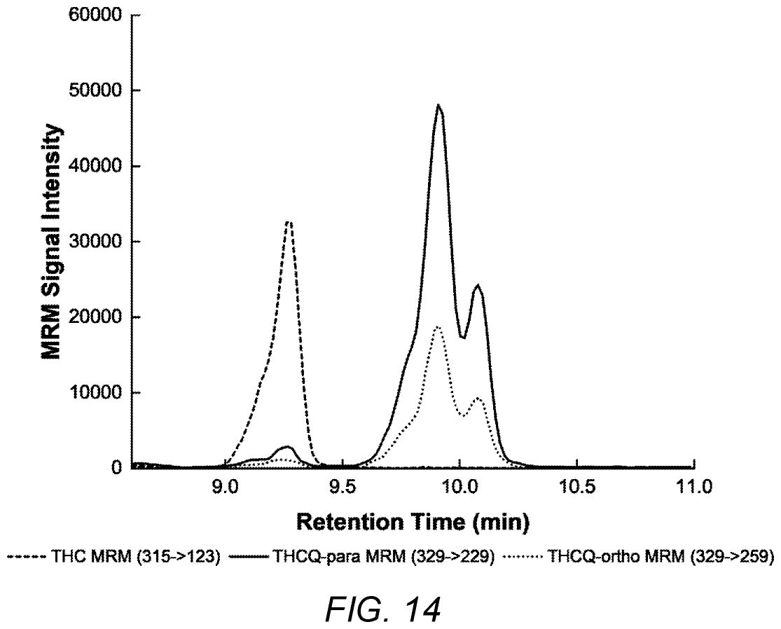

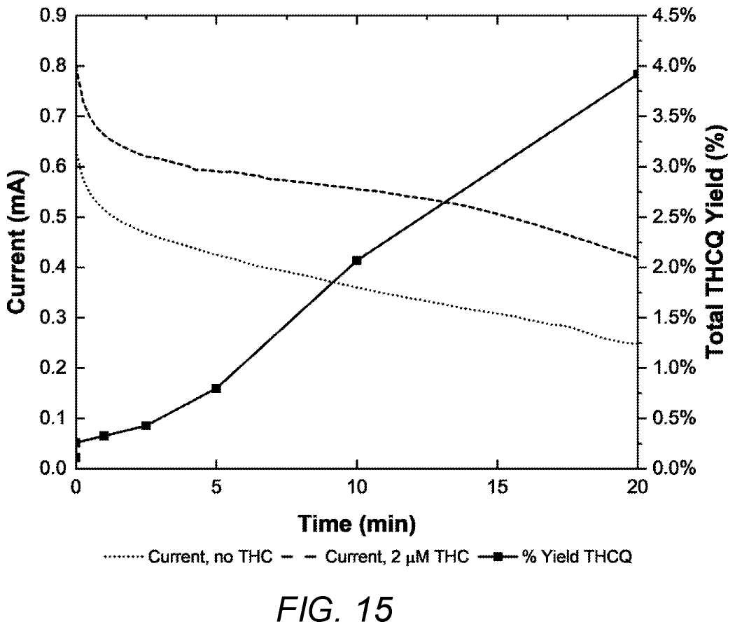

Phenol to quinone oxidation of THC remains operative in an H-Cell at low concentrations of THC. Several embodiments provide qualitatively the formation of THCQ at THC concentrations from about 0.1 μM to about 2 mM; or from about 0.1 to about 2 μM; or from about 2 μM to about 2 mM. Using the H-Cell conditions from entry 11 of Table 14, many embodiments provide a series of chronoamperometry results in relation to THCQ conversion. FIG. 14 illustrates an LC-MS/MS chromatogram of THC and THCQ, as both p-/o-THCQ isomers, in accordance with an embodiment. At a concentration of about 2 μM THC, a bias potential of about 0 V vs Ag/Ag⁺ can be applied to the THC solution, and the conversion of THC to THCQ can be observed as shown in FIG. 14. FIG. 15 illustrates chronoamperometry result with and without the presence of THC in the fuel cell in accordance with an embodiment. FIG. 15 shows the increase of the THCQ yield as time. FIG. 15 illustrates the measured increase in THCQ as current results are recorded with a bias potential of about 0 V vs Ag/Ag⁺.

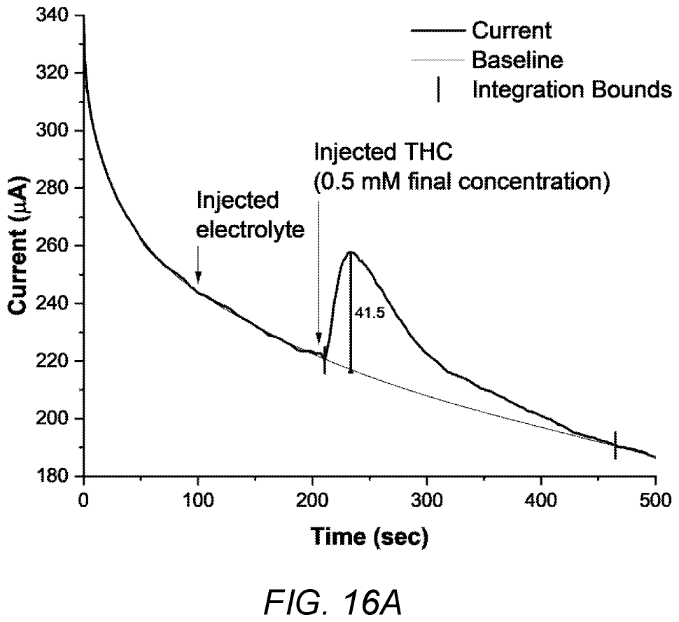

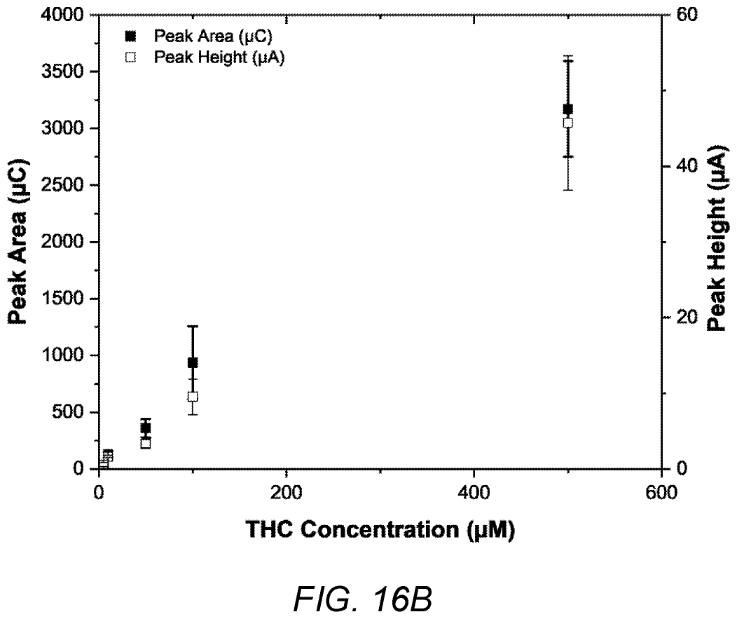

THC can be detected and monitored real-time using fuel cells. In several embodiments, the signals from THC oxidation can be proportional to the input THC concentration. A number of embodiments provide THC fuel cells can be integrated into breathalyzers for THC detection. Chronoamperometry results of THC fuel cells in accordance with an embodiment are illustrated in FIGS. 16A and 16B. FIG. 16A illustrates current (μA) of chronoamperometry measurements. At about 100 seconds, about 22 μL of electrolyte solution with 0 mM THC is added to the fuel cell and no signal response can be observed. At about 200 seconds, about 22 μL solution of about 159 mM THC in electrolyte is added to the fuel cell resulting in a final concentration of about 500 μM THC. The injection of THC generates an increase in peak current of about 41.5 μA from the baseline (FIG. 16A). FIG. 16B illustrates peak integration of total charge (μC) of chronoamperometry measurements. The addition of about 500 μM THC to the fuel cell generates a total charge by integrating the current over time with respect to baseline of about 3.31×10³ μC. THC concentration from about 5 μM, 10 μM, 50 μM, 100 μM, 500 μM, and 1000 μM can be added to the fuel cell and record their peak current and total charge. Each data point in FIG. 16B is an average of three measurements at each THC concentration. FIG. 16B shows a linear relationship of total current or total charge and input THC concentration until about 500 μM.

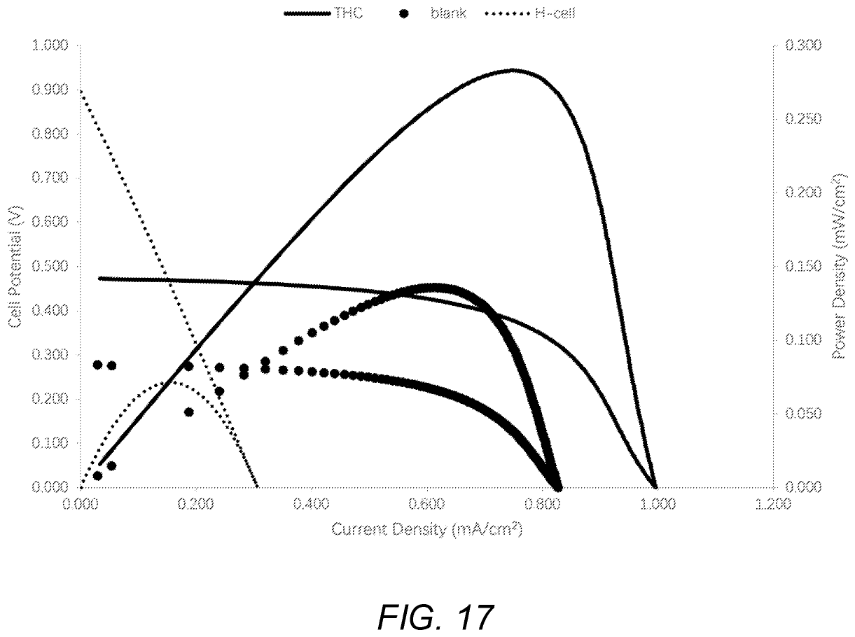

Many embodiments provide THC fuel cell stacks for THC detection. THC fuel cell stacks in accordance with several embodiments may eliminate the use of individual catholyte and/or anolyte in the fuel cell. The cathodes and/or anodes can be in a form of thin films, and can be made with textiles or printed on a substrate in accordance with certain embodiments. The ion exchange membrane can be sandwiched between the cathode and the anode layers to establish connection. The membrane may be hydrated to keep ion flow. Gas supply can be applied directly to the cathodes and/or anodes. FIG. 17 illustrates the comparison of performances of H-cell and fuel cell stack in accordance with an embodiment. The fuel cell stack can improve the readout signal of THC oxidation of at least 8 times.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately,” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

1. A method of oxidizing cannabinoid with a fuel cell comprising:
obtaining a sample from a source;
oxidizing the sample electrochemically using a fuel cell;
analyzing at least one signal generated during the oxidation of the sample selected from the group consisting of current, power, current density, power density, and charge; and
identifying if the cannabinoid is present based on the analysis.

2. The method of claim 1, wherein the sample is either in liquid phase or in gas phase.

3. The method of claim 1, wherein the sample is a biological sample extracted from an individual and the biological sample is biofluid, tear, saliva, mucus, urine, sweat, blood, or plasma.

4. The method of claim 1, wherein the sample is in gas phase and the sample is breath.

5. The method of claim 1, wherein the fuel cell comprises at least one electrolyte comprising at least one electrolyte salt selected from the group consisting of NBu₄PF₆, NEt₄PF₆, LiPF₆, LiPF₄, NBu₄BF₄, NEt₄BF₄, NBu₄ClO₄, and LiClO₄, dissolved in a solvent selected from the group consisting of an aqueous solvent, an organic solvent, and a mixture of an aqueous solvent and an organic solvent.

6. The method of claim 1, wherein the fuel cell comprises at least one solid electrolyte.

7. The method of claim 5, wherein the at least one electrolyte has a concentration from 0.01 M to 1 M, and the solvent has a volume fraction from 96% to 100%.

8. The method of claim 1, wherein the fuel cell comprises a cathode comprising a material selected from the group consisting of a transition metal, a metal oxide, a metal, and a metal alloy.

9. The method of claim 8, wherein the cathode is supported on a material selected from the group consisting of carbon, carbon black, carbon powder, carbon black powder, graphene, graphite, fullerene, nanotube, and carbon nanotube.

10. The method of claim 1, wherein the fuel cell comprises a cathode selected from the group consisting of platinum on carbon cloth, platinum on carbon paper, and platinum and ruthenium on carbon cloth.

11. The method of claim 1, wherein the fuel cell comprises an anode comprising a material selected from the group consisting of a transition metal, a metal oxide, a metal, and a metal alloy.

12. The method of claim 11, wherein the anode is supported on a material selected from the group consisting of carbon, carbon black, carbon powder, carbon black powder, graphene, graphite, fullerene, nanotube, and carbon nanotube.

13. The method of claim 1, wherein the fuel cell comprises an anode selected from the group consisting of Ni(OH)₂, Ni(OH)₂ modified with multi-wall carbon nanotubes (MWCNTs), CuO, CuO modified with MWCNTs, glassy carbon electrode, Cu on a carbon support, Pd on a carbon support, Pt on a carbon support, Fe on a carbon support, Pd on a carbon support, Rh on a carbon support, Ni on a carbon support, Ru on a carbon support, Pt and Ni on a carbon support, and Ni(OH)₂ on a carbon support.

14. The method of claim 13, wherein the carbon support is selected from the group consisting of: carbon black, carbon black XC-72, Vulcan XC72, Vulcan XC72R, carbon black powder, and Super P® carbon black powder.

15. The method of claim 1, wherein the fuel cell comprises a platinum on carbon cloth cathode and a Ru on a carbon support anode; or a carbon cloth cathode and a Ni(OH)₂ modified with MWCNTs anode; or a carbon cloth cathode and a CuO modified with MWCNTs anode; or a carbon cloth cathode and a Ru on Vulcan XC72 anode; or a carbon cloth cathode and a Pt on Vulcan XC72 anode.

16. The method of claim 1, wherein the fuel cell comprises an ion exchange membrane or a proton conducting membrane.

17. The method of claim 16, wherein the ion exchange membrane is selected from the group consisting of Nafion® 117, Nafion® 112, Nafion® 212, Xion® PEM, Fumasep® F930, Fumasep® FKB-PK-130, Fumasep® F950, Fumasep® FS950, Fumasep® FKE-50, and Fumasep® FAS-30.

18. The method of claim 1, wherein the fuel cell is a H-cell, a flow cell, or a stack cell.

19. The method of claim 1, wherein the fuel cell is configured to be integrated in a breathalyzer.

20. The method of claim 1, wherein the identification is in real-time.

21. The method of claim 1, wherein the cannabinoid is selected from the group consisting of Δ⁹-THC, Δ⁸-THC, CBN, and CBD.

22. The method of claim 1, wherein the fuel cell is part of an energy production process.

23. The method of claim 1, further comprising calibrating the fuel cell to establish a base line signal.

24. The method of claim 1, wherein the identification of cannabinoid outputs a cannabinoid concentration in the sample.

25. The method of claim 24, wherein the at least one signal has a linear relationship with the cannabinoid concentration.

26. The method of claim 1, wherein the cannabinoid is Δ⁹-THC and the oxidized sample is Δ⁹-THCQ.

27. A cannabinoid fuel cell comprising:
a cathode;
an anode;
an ion exchange membrane; and
an electrolyte;
wherein the ion exchange membrane is disposed between the cathode and the anode, and the electrolyte is in contact with the anode; and
wherein the fuel cell is configured to oxidize a sample electrochemically; analyze at least one signal generated during an oxidation process of the sample selected from the group consisting of current, power, current density, power density, and charge; and output a cannabinoid concentration from the sample.

28. The fuel cell of claim 27, wherein the sample is either in liquid phase or in gas phase.

29. The fuel cell of claim 27, wherein the sample is a biological sample extracted from an individual and the biological sample is biofluid, tear, saliva, mucus, urine, sweat, blood, or plasma.

30. The fuel cell of claim 27, wherein the sample is in gas phase and the sample is breath.

31. The fuel cell of claim 27, wherein the electrolyte comprises at least one electrolyte salt selected from the group consisting of NBu₄PF₆, NEt₄PF₆, LiPF₆, LiPF₄, NBu₄BF₄, NEt₄BF₄, NBu₄ClO₄, and LiClO₄, dissolved in a solvent selected from the group consisting of an aqueous solvent, an organic solvent, and a mixture of an aqueous solvent and an organic solvent.

32. The fuel cell of claim 27, wherein the electrolyte is a solid electrolyte.

33. The fuel cell of claim 31, wherein the electrolyte has a concentration from 0.01 M to 1 M, and the solvent has a volume fraction from 96% to 100%.

34. The fuel cell of claim 27, wherein the cathode comprises a material selected from the group consisting of a transition metal, a metal oxide, a metal, and a metal alloy.

35. The fuel cell of claim 34, wherein the cathode is supported on a material selected from the group consisting of carbon, carbon black, carbon powder, carbon black powder, graphene, graphite, fullerene, nanotube, and carbon nanotube.

36. The fuel cell of claim 27, wherein the cathode is selected from the group consisting of platinum on carbon cloth, platinum on carbon paper, and platinum and ruthenium on carbon cloth.

37. The fuel cell of claim 27, wherein the anode comprises a material selected from the group consisting of a transition metal, a metal oxide, a metal, and a metal alloy.

38. The fuel cell of claim 37, wherein the anode is supported on a material selected from the group consisting of carbon, carbon black, carbon powder, carbon black powder, graphene, graphite, fullerene, nanotube, and carbon nanotube.

39. The fuel cell of claim 27, wherein the fuel cell comprises an anode selected from the group consisting of Ni(OH)₂, Ni(OH)₂ modified with multi-wall carbon nanotubes (MWCNTs), CuO, CuO modified with MWCNTs, glassy carbon electrode, Cu on a carbon support, Pd on a carbon support, Pt on a carbon support, Fe on a carbon support, Pd on a carbon support, Rh on a carbon support, Ni on a carbon support, Ru on a carbon support, Pt and Ni on a carbon support, and Ni(OH)₂ on a carbon support.

40. The fuel cell of claim 39, wherein the carbon support is selected from the group consisting of: carbon black, carbon black XC-72, Vulcan XC72, Vulcan XC72R, carbon black powder, and Super P® carbon black powder.

41. The fuel cell of claim 27, wherein the cathode is a platinum on carbon cloth and the anode is Ru on a carbon support; or the cathode is carbon cloth and the anode is Ni(OH)₂ modified with MWCNTs; or the cathode is carbon cloth and the anode is CuO modified with MWCNTs; or the cathode is carbon cloth and the anode is Ru on Vulcan XC72; or the cathode is carbon cloth and the anode is Pt on Vulcan XC72.

42. The fuel cell of claim 27, wherein the ion exchange membrane is a proton conducting membrane.

43. The fuel cell of claim 27, wherein the ion exchange membrane is selected from the group consisting of Nafion® 117, Nafion® 112, Nafion® 212, Xion® PEM, Fumasep® F930, Fumasep® FKB-PK-130, Fumasep® F950, Fumasep® FS950, Fumasep® FKE-50, and Fumasep® FAS-30.

44. The fuel cell of claim 27, wherein the fuel cell is a H-cell, a flow cell, or a stack cell.

45. The fuel cell of claim 27, wherein the fuel cell is configured to be integrated in a breathalyzer.

46. The fuel cell of claim 27, wherein the fuel cell outputs the cannabinoid concentration in real-time.

47. The fuel cell of claim 27, wherein the cannabinoid is selected from the group consisting of Δ⁹-THC, Δ⁸-THC, CBN, and CBD.

48. The fuel cell of claim 27, wherein the fuel cell is part of an energy production process.

49. The fuel cell of claim 27, further comprising a computer system to analyze the at least one signal of the oxidized sample.

50. The fuel cell of claim 27, wherein the at least one signal has a linear relationship with the cannabinoid concentration.

51. The fuel cell of claim 27, further comprising an anode gas diffusion layer, an anode flow plate, an anode current collector, an anode end plate, a cathode gas diffusion layer, a cathode flow plate, a cathode current collector, and a cathode end plate.

52. The fuel cell of claim 27, wherein the cannabinoid is Δ⁹-THC and the oxidized sample is Δ⁹-THCQ.
